SEQUENCE LISTING (55 sequences)

SEQ ID NO: 1 - protein, 5 aa, synthetic construct
  REGION 1..5: note = Synthetic: ZY0EPQ-E02; CHDR1
GYYWN

SEQ ID NO: 2 - protein, 16 aa, synthetic construct
  REGION 1..16: note = Synthetic: ZY0EPQ-E02; CHDR2
EINHSGSTNYNPSLKS

SEQ ID NO: 3 - protein, 9 aa, synthetic construct
  REGION 1..9: note = Synthetic: ZY0EPQ-E02; CHDR3
NLYNWNLDS

SEQ ID NO: 4 - protein, 11 aa, synthetic construct
  REGION 1..11: note = Synthetic: ZY0EPQ-E02; CLDR1
RASQGIRNDLG

SEQ ID NO: 5 - protein, 7 aa, synthetic construct
  REGION 1..7: note = Synthetic: ZY0EPQ-E02; CLDR2
VASSLQS

SEQ ID NO: 6 - protein, 9 aa, synthetic construct
  REGION 1..9: note = Synthetic: ZY0EPQ-E02; CLDR3
LQHNSYPRT

SEQ ID NO: 7 - protein, 5 aa, synthetic construct
  REGION 1..5: note = Synthetic: ZY0EQD-E02; CHDR1
GYYWN

SEQ ID NO: 8 - protein, 16 aa, synthetic construct
  REGION 1..16: note = Synthetic: ZY0EQD-E02; CHDR2
EINHSGSTSYNPSLKS

SEQ ID NO: 9 - protein, 9 aa, synthetic construct
  REGION 1..9: note = Synthetic: ZY0EQD-E02; CHDR3
VLYNWNVDS

SEQ ID NO: 10 - protein, 11 aa, synthetic construct
  REGION 1..11: note = Synthetic: ZY0EQD-E02; CLDR1
RASQDIRNDVG

SEQ ID NO: 11 - protein, 7 aa, synthetic construct
  REGION 1..7: note = Synthetic: ZY0EQD-E02; CLDR2
AASRLQS

SEQ ID NO: 12 - protein, 9 aa, synthetic construct
  REGION 1..9: note = Synthetic: ZY0EQD-E02; CLDR3
LQHNSYPRT

SEQ ID NO: 13 - protein, 7 aa, synthetic construct
  REGION 1..7: note = Synthetic: ZY0EOB-F05; CHDR1
SGGYYWS

SEQ ID NO: 14 - protein, 16 aa, synthetic construct
  REGION 1..16: note = Synthetic: ZY0EOB-F05; CHDR2
NIYYSGSTYYNPSLKS

SEQ ID NO: 15 - protein, 20 aa, synthetic construct
  REGION 1..20: note = Synthetic: ZY0EOB-F05; CHDR3
EKALATVTPSGYENYYTVDV

SEQ ID NO: 16 - protein, 11 aa, synthetic construct
  REGION 1..11: note = Synthetic: ZY0EOB-F05; CLDR1
WASQGISSYLA

SEQ ID NO: 17 - protein, 7 aa, synthetic construct
  REGION 1..7: note = Synthetic: ZY0EOB-F05; CLDR2
AASTLQS

SEQ ID NO: 18 - protein, 9 aa, synthetic construct
  REGION 1..9: note = Synthetic: ZY0EOB-F05; CLDR3
QHLNSYPLT

SEQ ID NO: 19 - protein, 7 aa, synthetic construct
  REGION 1..7: note = Synthetic: ZY0EO5-E07; CHDR1
SGGYYWS

SEQ ID NO: 20 - protein, 16 aa, synthetic construct
  REGION 1..16: note = Synthetic: ZY0EO5-E07; CHDR2
NIYYSGSTYYNPSLKS

SEQ ID NO: 21 - protein, 20 aa, synthetic construct
  REGION 1..20: note = Synthetic: ZY0EO5-E07; CHDR3
EKALASVIPSGYENYYVVDV

SEQ ID NO: 22 - protein, 11 aa, synthetic construct
  REGION 1..11: note = Synthetic: ZY0EO5-E07; CLDR1
WASQGIAGYLA

SEQ ID NO: 23 - protein, 7 aa, synthetic construct
  REGION 1..7: note = Synthetic: ZY0EO5-E07; CLDR2
AASTLQS

SEQ ID NO: 24 - protein, 9 aa, synthetic construct
  REGION 1..9: note = Synthetic: ZY0EO5-E07; CLDR3
QHLNSYPLT

SEQ ID NO: 25 - protein, 5 aa, synthetic construct
  REGION 1..5: note = Synthetic: ZY0EP0-C07; CHDR1
DYYMS

SEQ ID NO: 26 - protein, 17 aa, synthetic construct
  REGION 1..17: note = Synthetic: ZY0EP0-C07; CHDR2
YISSSGSTIYYTDSVKG

SEQ ID NO: 27 - protein, 7 aa, synthetic construct
  REGION 1..7: note = Synthetic: ZY0EP0-C07; CHDR3
DGVGFDY

SEQ ID NO: 28 - protein, 12 aa, synthetic construct
  REGION 1..12: note = Synthetic: ZY0EP0-C07; CLDR1
RASQSVSSSYLA

SEQ ID NO: 29 - protein, 7 aa, synthetic construct
  REGION 1..7: note = Synthetic: ZY0EP0-C07; CLDR2
AASSRAT

SEQ ID NO: 30 - protein, 10 aa, synthetic construct
  REGION 1..10: note = Synthetic: ZY0EP0-C07; CLDR3
QQYGSSPLYT

SEQ ID NO: 31 - protein, 117 aa, synthetic construct
  REGION 1..117: note = Synthetic: ZY0EPQ-E02, variable heavy chain
QVQLQQWGAGLLKPSETLSLTCAVYGGSFSGYYWNWIRQPPGKGLEWIGEINHSGSTNYNPSLKSRVTILVDTSKNQFSLKLSSVTAADTAVYYCARNLYNWNLDSWGQGTLVTVSS

SEQ ID NO: 32 - protein, 107 aa, synthetic construct
  REGION 1..107: note = Synthetic: ZY0EPQ-E02, variable light chain
DIQMTQSPSSLSASVGDRVTITCRASQGIRNDLGWYQQKPGRAPKRLIYVASSLQSGVPSRFSGSGSGTEFTLTISSLQPEDFATYYCLQHNSYPRTFGQGTKVEIK

SEQ ID NO: 33 - protein, 117 aa, synthetic construct
  REGION 1..117: note = Synthetic: ZY0EQD-E02, variable heavy chain, e.g., pre-germlining
QVQLQQWGAGLLKPSETLSLTCAVYGGSFSGYYWNWIRQPPGKGLEWIGEINHSGSTSYNPSLKSRVTISVDTSKNQFSLKLSSVTAADTAVYYCARVLYNWNVDSWGQGTLVTVSS

SEQ ID NO: 34 - protein, 107 aa, synthetic construct
  REGION 1..107: note = Synthetic: ZY0EQD-E02, variable light chain
DIQMTQSPSSLSASVGDRVTITCRASQDIRNDVGWYQQKPGKAPKRLIYAASRLQSGVPSRFSGSGSGTEFTLTISSLQPEDFATYYCLQHNSYPRTFGQGTKVEIK

SEQ ID NO: 35 - protein, 130 aa, synthetic construct
  REGION 1..130: note = Synthetic: ZY0EOB-F05, variable heavy chain
QVQLQESGPGLVKPSQTLSLTCTVSDGSISSGGYYWSWIRQHPGKGLEWIGNIYYSGSTYYNPSLKSRVTISVDTSKNQFSLKLNSVTAADTAVYYCATEKALATVTPSGYENYYTVDVWGQGTTVTVSS

SEQ ID NO: 36 - protein, 107 aa, synthetic construct
  REGION 1..107: note = Synthetic: ZY0EOB-F05, variable light chain
DIQLTQSPSFLSASVGDRVTITCWASQGISSYLAWYQQKPGKAPKLLIYAASTLQSGVPSRFSGSGSGTEFTLTISSLQPEDFATYYCQHLNSYPLTFGGGTKVEIK

SEQ ID NO: 37 - protein, 130 aa, synthetic construct
  REGION 1..130: note = Synthetic: ZY0EO5-E07, variable heavy chain
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCAREKALASVIPSGYENYYVVDVWGQGTTVTVSS

SEQ ID NO: 38 - protein, 107 aa, synthetic construct
  REGION 1..107: note = Synthetic: ZY0EO5-E07, variable light chain
DIQLTQSPSFLSASVGGRVTITCWASQGIAGYLAWYQQKPGKAPKLLIYAASTLQSGVPSRFSGSGSGTEFTLTISSLQPEDFATYYCQHLNSYPLTFGGGTKVEIK

SEQ ID NO: 39 - protein, 116 aa, synthetic construct
  REGION 1..116: note = Synthetic: ZY0EP0-C07, variable heavy chain
QVQLVESGGVLVKPGGSLRLSCAASGFTLSDYYMSWIRQAPGMGLEWVSYISSSGSTIYYTDSVKGRFTISRDSAKNSLYLQMNSLRAEDTAVYYCARDGVGFDYWGQGTLVTVSS

SEQ ID NO: 40 - protein, 109 aa, synthetic construct
  REGION 1..109: note = Synthetic: ZY0EP0-C07, variable light chain
EIVLTQSPGTLSLFPGERATLSCRASQSVSSSYLAWYQQKPGQSPRLLIYAASSRATGIPDRFSGSGSGTDFTLTISRLEPEDFAVYYCQQYGSSPLYTFGQGTKLEIK

SEQ ID NO: 41 - protein, 331 aa, synthetic construct
  REGION 1..331: note = Synthetic: Maia heavy chain constant region, cysteine insertion underlined
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 107 aa, synthetic construct [sequence omitted]

SEQ ID NO: 43 - protein, 117 aa, synthetic construct
  REGION 1..117: note = Synthetic: ZY0EQD-E02, variable heavy chain, e.g., pre-germlining, e.g., variant of SEQ ID NO: 33/ SEQ ID NO: 45
QVQLQQWGAGLLKPSETLSLTCTVYGGSFSGYYWNWIRQPPGRGLEWIGEINHSGSTSYNPSLKSRITISIDTSKNQFSLKLSSVTAADTAVYYCARVLYNWNVDSWGQGTLVTVSS

SEQ ID NO: 44 - protein, 214 aa, synthetic construct
  REGION 1..214: note = Synthetic: ZY0EQD-E02, light chain
DIQMTQSPSSLSASVGDRVTITCRASQDIRNDVGWYQQKPGKAPKRLIYAASRLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 117 aa, synthetic construct
  REGION 1..117: note = Synthetic: EQD-E02_GL, variable heavy chain, GL = germlined
QVQLQQWGAGLLKPSETLSLACTVYGGSFSGYYWNWIRQPPGKGLEWIGEINHSGSTSYNPSLKSRVTISVDTSKNQFSLKLSSVTAADTAVYYCARVLYNWNVDSWGQGTLVTVSS

SEQ ID NO: 46 - protein, 117 aa, synthetic construct
  REGION 1..117: note = Synthetic: EQD-E02-GLY, variable heavy chain, GLY = germlined with a Y substitution
QVQLQQWGAGLLKPSETLSLACTVYGGSFSGYYWNWIRQPPGKGLEWIGEIYHSGSTSYNPSLKSRVTISVDTSKNQFSLKLSSVTAADTAVYYCARVLYNWNVDSWGQGTLVTVSS

SEQ ID NO: 47 - protein, 117 aa, synthetic construct
  REGION 1..117: note = Synthetic: EQD-E02-GLQ, variable heavy chain, GLQ = germlined with a Q substitution
QVQLQQWGAGLLKPSETLSLACTVYGGSFSGYYWNWIRQPPGKGLEWIGEIQHSGSTSYNPSLKSRVTISVDTSKNQFSLKLSSVTAADTAVYYCARVLYNWNVDSWGQGTLVTVSS

SEQ ID NO: 48 - protein, 448 aa, synthetic construct
  REGION 1..448: note = Synthetic: E02-GL-Maia-heavy chain, cysteine insertion underlined
QVQLQQWGAGLLKPSETLSLACTVYGGSFSGYYWNWIRQPPGKGLEWIGEINHSGSTSYNPSLKSRVTISVDTSKNQFSLKLSSVTAADTAVYYCARVLYNWNVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 448 aa, synthetic construct
  REGION 1..448: note = Synthetic: E02-GLY-Maia-heavy chain, GLY = germlined with a Y substitution
QVQLQQWGAGLLKPSETLSLACTVYGGSFSGYYWNWIRQPPGKGLEWIGEIYHSGSTSYNPSLKSRVTISVDTSKNQFSLKLSSVTAADTAVYYCARVLYNWNVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 448 aa, synthetic construct
  REGION 1..448: note = Synthetic: E02-GLQ-Maia-heavy chain, GLQ = germlined with a Q substitution
QVQLQQWGAGLLKPSETLSLACTVYGGSFSGYYWNWIRQPPGKGLEWIGEIQHSGSTSYNPSLKSRVTISVDTSKNQFSLKLSSVTAADTAVYYCARVLYNWNVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 447 aa, synthetic construct
  REGION 1..447: note = Synthetic: E02-GL-WT-heavy chain
QVQLQQWGAGLLKPSETLSLACTVYGGSFSGYYWNWIRQPPGKGLEWIGEINHSGSTSYNPSLKSRVTISVDTSKNQFSLKLSSVTAADTAVYYCARVLYNWNVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 331 aa, synthetic construct; identical to SEQ ID NO: 41
  REGION 1..331: note = Synthetic: heavy chain constant region

SEQ ID NO: 53 - DNA, 858 bp, Homo sapiens
  misc_feature 1..858: note = Human B7H4 nucleic acid sequence, including 5' and 3' UTR
  source 1..858: mol_type = genomic DNA
gccaccatggcttccctggggcagatcctcttctggagcataattagcatcatcattattctggctggagcaattgcactcatcattggctttggtatttcagggagacactccatcacagtcactactgtcgcctcagctgggaacattggggaggatggaatcctgagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaataataa

SEQ ID NO: 54 - DNA, 846 bp, Homo sapiens
  misc_feature 1..846: note = Human B7H4 nucleic acid sequence, coding sequence
  source 1..846: mol_type = genomic DNA
atggcttccctggggcagatcctcttctggagcataattagcatcatcattattctggctggagcaattgcactcatcattggctttggtatttcagggagacactccatcacagtcactactgtcgcctcagctgggaacattggggaggatggaatcctgagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaa

SEQ ID NO: 55 - protein, 282 aa, Homo sapiens
  REGION 1..282: note = misc_feature - Human B7H4 polypeptide sequence; UniProt Accession No.: Q7Z7D3
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

(19) United States
(12) Patent Application Publication
(10) Pub. No.: US 2026/0295065 A1
(43) Pub. Date: Oct. 1, 2026

(54) B7-H4 THERAPEUTIC BINDING MOLECULES FOR THE TREATMENT OF CANCER

(71) Applicant: MEDIMMUNE LIMITED, Cambridge (GB)

(72) Inventors: Andreea VARGA, Cambridge (GB); Giulia FABBRI, Waltham, MA (US)

(21) Appl. No.: 19/478,530

(22) PCT Filed: Apr. 26, 2024
(86) PCT No.: PCT/EP2024/061668

§ 371 Date: Oct. 24, 2025

Related U.S. Application Data
(60) Provisional application No. 63/498,998, filed on Apr. 28, 2023.
(60) Provisional application No. 63/505,727, filed on Jun. 2, 2023.

(51) Int. Cl. A61K 47/68; A61P 35/00; G01N 33/575

(52) U.S. Cl. CPC A61K 47/6849; A61K 47/68037; A61P 35/00; G01N 33/5758; G01N 2333/70532; G01N 2474/20

(57) ABSTRACT

The disclosure relates to methods for treating cancer in a subject, comprising administering to the subject an anti-body-drug conjugate (ADC) that specifically binds to B7-H4. In aspects, the ADC is administered to the subject in an amount from about 0.8 mg/kg to about 4.8 mg/kg.

### CROSS-REFERENCE TO RELATED APPLICATIONS

This application claims priority benefit of U.S. Provisional Patent Application No. 63/498,998, filed Apr. 28, 2023, which is incorporated by reference herein in its entirety for all purposes. This application also claims priority benefit of U.S. Provisional Patent Application No. 63/505,727, filed Jun. 2, 2023, which is incorporated by reference herein in its entirety for all purposes.

### INCORPORATION-BY-REFERENCE OF MATERIAL SUBMITTED ELECTRONICALLY

This application incorporates by reference a Sequence Listing submitted with the application as an XML file entitled “B7H4-200-WO-PCT SL”, created on Apr. 12, 2024, and having a size of 76 kilobytes.

### FIELD

The present disclosure provides methods for treating cancer in a subject, comprising administering to the subject an antibody-drug conjugate (ADC) that specifically binds to B7-H4.

### BACKGROUND

Surgery, radiation therapy and chemotherapy, alone or in combination, are the most traditional and widely used treatment methods for cancer. While these treatment methods are effective in removing or killing cancer cells, they often result in undesirable side effects such as hair loss, anemia, severe nausea and death of healthy cells in patients treated. These limitations present an urgent need for innovative and less harmful treatment methods of cancer. Antibody based cancer therapies rely on the recognition and binding of antibody-drug conjugates to specific proteins on cancer cells. Antibody drug conjugates (ADCs) can take advantage of the specificity of the antibody portion of the conjugate to deliver a highly toxic agent directly to the cells to be killed. Using antibody or ADC cancer therapies alone or in combination with other small molecule based cancer therapies can improve treatment outcomes by attacking malignant cells and tumors in one or more than one way.

### SUMMARY

The present disclosure provides a method of treating cancer in a subject, comprising administering to the subject an antibody-drug conjugate (ADC) that specifically binds to B7-H4 in an amount from about 0.8 mg/kg to about 4.8 mg/kg. The ADC comprises:
i. an antibody or antigen binding fragment thereof which binds to a B7-H4 polypeptide, comprising:
   a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
   b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof; c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
   (d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
   (e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof;
ii. a cleavable linker; and
iii. a cytotoxic agent.

In some aspects, the amount of ADC administered is about 0.8 mg/kg, about 1.6 mg/kg, about 2.0 mg/kg, about 2.4 mg/kg, about 3.2 mg/kg, about 3.6 mg/kg or about 4.8 mg/kg. In some aspects, the amount of ADC administered is about 0.8 mg/kg, about 1.6 mg/kg, about 2.4 mg/kg, about 3.2 mg/kg, about 3.6 mg/kg or about 4.8 mg/kg. In some aspects, the amount of ADC administered is about 1.6 mg/kg. In some aspects, the amount of ADC administered is about 2.0 mg/kg. In some aspects, the amount of ADC administered is about 2.4 mg/kg. In some aspects, the amount of ADC administered is about 3.2 mg/kg.

In some aspects, the cancer comprises a cancer cell which expresses B7-H4.

In some aspects, the ADC is administered to the subject once every three weeks. In some aspects, the subject has been previously treated with another chemotherapeutic agent. In some aspects, the subject has been previously treated with a platinum-based chemotherapeutic agent.

In some aspects, the cancer is selected from endometrial cancer, ovarian cancer, biliary tract cancer, breast cancer, pancreatic cancer, prostate cancer, hematological cancer, cholangiocarcinoma, NSCLC (squamous and/or adenocarcinoma), gastrointestinal cancer such as gastric cancer and colorectal cancer, and lung cancer. In some aspects, the cancer is selected from endometrial cancer, ovarian cancer, breast cancer, pancreatic cancer, prostate cancer, hematological cancer, cholangiocarcinoma, NSCLC (squamous and/or adenocarcinoma), gastrointestinal cancer such as gastric cancer and colorectal cancer, and lung cancer. In some aspects, the cancer is a breast cancer selected from hormone receptor-positive (HR+) breast cancer, human epidermal growth factor receptor 2 positive (HER2+) breast cancer, and triple negative breast cancer (TNBC). In some aspects, the cancer is homologous recombination deficient (HRD) cancer. In some aspects, the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L. In some aspects, the mutation in an HRD gene is selected from BRCA1, BRCA2, and ATM.

In some aspects, the antibody or antigen binding fragment thereof comprises:
i. a variable heavy (VH) chain and a variable light (VL) chain comprising the amino acid sequence of SEQ ID NO: 45 and SEQ ID NO: 34, respectively, or a functional variant thereof;
ii. a variable heavy (VH) chain and a variable light (VL) chain comprising the amino acid sequence of SEQ ID NO: 33 and SEQ ID NO: 34, respectively, or a functional variant thereof;
iii. a variable heavy (VH) chain and a variable light (VL) chain comprising the amino acid sequence of SEQ ID NO: 43 and SEQ ID NO: 34, respectively, or a functional variant thereof;
iv. a variable heavy (VH) chain and a variable light (VL) chain comprising the amino acid sequence of SEQ ID NO: 46 and SEQ ID NO: 34, respectively, or a functional variant thereof;
v. a variable heavy (VH) chain and a variable light (VL) chain comprising the amino acid sequence of SEQ ID NO: 47 and SEQ ID NO: 34, respectively, or a functional variant thereof;
vi. a VH chain and a VL chain comprising the amino acid sequence of SEQ ID NO: 31, and SEQ ID NO: 32, respectively, or a functional variant thereof;
vii. a VH chain and a VL chain comprising the amino acid sequence of SEQ ID NO: 35 and SEQ ID NO: 36, respectively, or a functional variant thereof;
viii. a VH chain and a VL chain comprising the amino acid sequence of SEQ ID NO: 37 and SEQ ID NO: 38, respectively, or a functional variant thereof; or
ix, a VH chain and a VL chain comprising the amino acid sequence of SEQ ID NO: 39 and SEQ ID NO: 40, respectively, or a functional variant thereof.

In some aspects, the antibody or antigen binding fragment thereof comprises: a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof.

In some aspects, the antibody or antigen binding fragment thereof comprises a VH chain and a VL chain comprising the amino acid sequence of SEQ ID NO: 45 and SEQ ID NO: 34, respectively, or a functional variant thereof.

In some aspects, the antibody or antigen binding fragment thereof binds an OVCAR4 cell line.

In some aspects, the antibody or antigen binding fragment thereof comprises a heavy chain constant region comprising the amino acid sequence of SEQ ID NO: 41. In some aspects, the antibody or antigen binding fragment thereof comprises a heavy chain constant region comprising the amino acid sequence of SEQ ID NO: 52. In some aspects, the antibody or antigen binding fragment thereof comprises a light chain constant region comprising the amino acid sequence of SEQ ID NO: 42.

In some aspects, the antibody or antigen binding fragment thereof comprises a heavy chain comprising the amino acid sequence of SEQ ID NO: 51; and a light chain comprising the amino acid sequence of SEQ ID NO: 44. In some aspects, the antibody or antigen binding fragment thereof comprises a heavy chain comprising the amino acid sequence of SEQ ID NO: 48; and a light chain comprising the amino acid sequence of SEQ ID NO: 44.

In some aspects, the antibody or antigen binding fragment thereof is a monoclonal antibody. In some aspects, the antibody or antigen binding fragment thereof is a humanized monoclonal antibody.

In some aspects, the cleavable linker is an mp-PEG8-val-ala linker.

In some aspects, the cytotoxic agent is a topoisomerase inhibitor.

In some aspects, the topoisomerase inhibitor is a compound of Formula A*

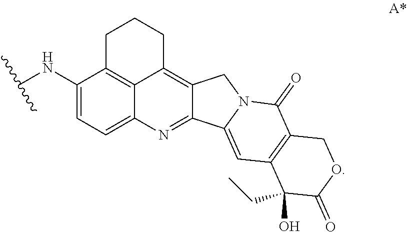 [SMILES: *Nc1ccc2nc3c(c4c2c1CCC4)Cn1c-3cc2c(c1=O)COC(=O)[C@]2(O)CC]

In some aspects, the ii) cleavable linker and iii) cytotoxic agent together are selected from the following compounds:

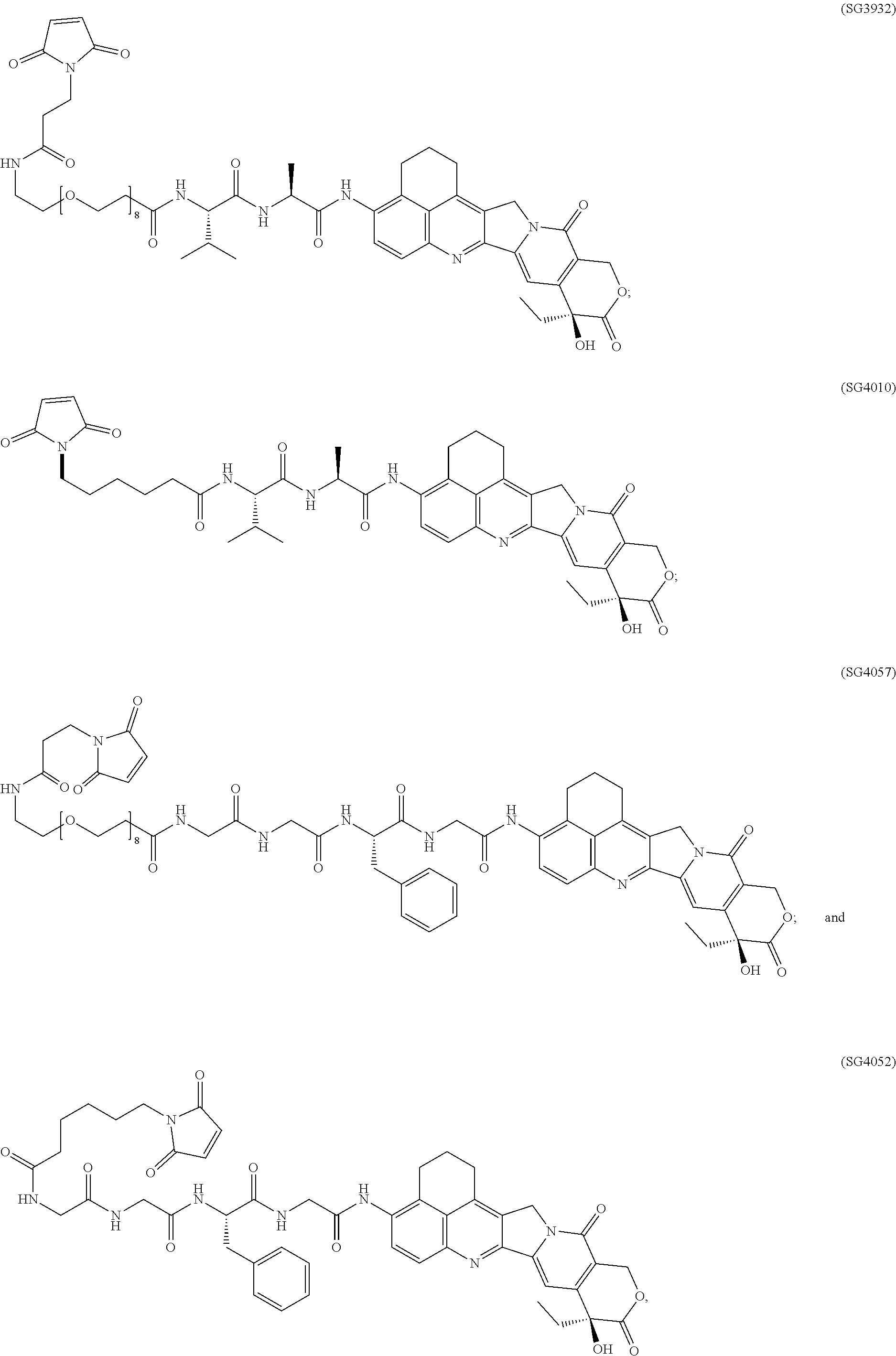

In some aspects, the ii) linker and iii) cytotoxic agent together are the compound SG3932.

In some aspects, the ADC has a drug to antibody ratio (DAR) of between about 1 and about 8. In some aspects, the ADC has a DAR of about 8.

In some aspects, the present disclosure further provides a method of treating cancer in a subject, comprising administering to the subject an antibody-drug conjugate (ADC) that specifically binds to B7-H4 in an amount from about 0.8 mg/kg to about 4.8 mg/kg, the ADC comprising:
i. an antibody or antigen binding fragment thereof which binds to a B7-H4 polypeptide, comprising: a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof; and
ii. a cleavable linker and cytotoxic agent conjugated to the antibody or antigen binding fragment thereof having the formula:

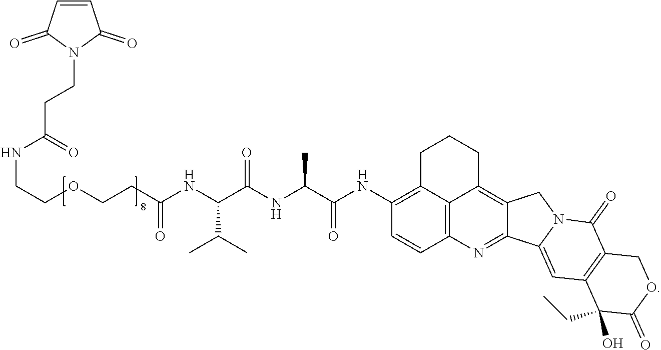

In some aspects, the amount of ADC administered is about 0.8 mg/kg, about 1.6 mg/kg, about 2.0 mg/kg, about 2.4 mg/kg, about 3.2 mg/kg, about 3.6 mg/kg or about 4.8 mg/kg. In some aspects, the amount of ADC administered is about 0.8 mg/kg, about 1.6 mg/kg, about 2.4 mg/kg, about 3.2 mg/kg, about 3.6 mg/kg or about 4.8 mg/kg. In some aspects, the amount of ADC administered is about 1.6 mg/kg. In some aspects, the amount of ADC administered is about 2.0 mg/kg. In some aspects, the amount of ADC administered is about 2.4 mg/kg. In some aspects, the amount of ADC administered is about 3.2 mg/kg.

In some aspects, the antibody or antigen binding fragment thereof comprises a VH chain and a VL chain comprising the amino acid sequence of SEQ ID NO: 45 and SEQ ID NO: 34, respectively, or a functional variant thereof.

In some aspects, the cancer comprises a cancer cell which expresses B7-H4.

In some aspects, the ADC is administered to the subject once every three weeks. In some aspects, the subject has been previously treated with another chemotherapeutic agent. In some aspects, the subject has been previously treated with a platinum-based chemotherapeutic agent.

In some aspects, the cancer is selected from endometrial cancer, ovarian cancer, biliary tract cancer, breast cancer, pancreatic cancer, prostate cancer, hematological cancer, cholangiocarcinoma, NSCLC (squamous and/or adenocarcinoma), gastrointestinal cancer such as gastric cancer and colorectal cancer, and lung cancer. In some aspects, the cancer is selected from endometrial cancer, ovarian cancer, breast cancer, pancreatic cancer, prostate cancer, hematological cancer, cholangiocarcinoma, NSCLC (squamous and/or adenocarcinoma), gastrointestinal cancer such as gastric cancer and colorectal cancer, and lung cancer.

In some aspects, the cancer is a breast cancer selected from hormone receptor-positive (HR+) breast cancer, human epidermal growth factor receptor 2 positive (HER2+) breast cancer, and triple negative breast cancer (TNBC).

In some aspects, the cancer is homologous recombination deficient (HRD) cancer.

In some aspects, the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L.

In some aspects, the mutation in an HRD gene is selected from BRCA1, BRCA2, and ATM.

In some aspects of the above methods, at least about 25% of cancer cells in the subject are B7-H4 positive cells. In some aspects of the above methods, the B7-H4 positive cells are assayed using immunohistochemistry (IHC).

In some aspects, the present disclosure further provides a pharmaceutical composition comprising an antibody-drug conjugate (ADC) that specifically binds to B7-H4 which is administered in an amount from about 0.8 mg/kg to about 4.8 mg/kg, the ADC comprising:
i. an antibody or antigen binding fragment thereof which binds to a B7-H4 polypeptide, comprising: a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof; and
ii. a cleavable linker and cytotoxic agent conjugated to the antibody or antigen binding fragment thereof having the formula:

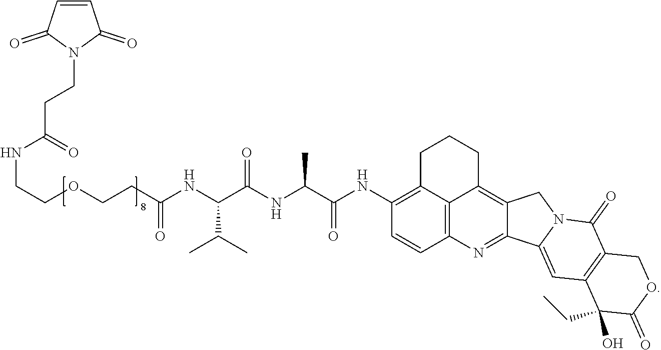

In some aspects, the pharmaceutical composition is administered in an amount from about 0.8 mg/kg, about 1.6 mg/kg, about 2.0 mg/kg, about 2.4 mg/kg, about 3.2 mg/kg, about 3.6 mg/kg or about 4.8 mg/kg ADC. In some aspects, the pharmaceutical composition is administered in an amount from about 0.8 mg/kg, about 1.6 mg/kg, about 2.4 mg/kg, about 3.2 mg/kg, about 3.6 mg/kg or about 4.8 mg/kg ADC. In some aspects, the pharmaceutical composition is administered in an amount of about 1.6 mg/kg ADC. In some aspects, the pharmaceutical composition is administered in an amount of about 2.0 mg/kg ADC. In some aspects, the pharmaceutical composition is administered in an amount of about 2.4 mg/kg ADC. In some aspects, the pharmaceutical composition is administered in an amount of about 3.2 mg/kg ADC.

In some aspects, the antibody or antigen binding fragment of the ADC comprises a VH chain and a VL chain comprising the amino acid sequence of SEQ ID NO: 45 and SEQ ID NO: 34, respectively, or a functional variant thereof.

In some aspects, the present disclosure further provides a pharmaceutical composition comprising an antibody-drug conjugate (ADC) that specifically binds to B7-H4 which is administered in an amount from about 25 mg to about 900 mg, the ADC comprising:
i. an antibody or antigen binding fragment thereof which binds to a B7-H4 polypeptide, comprising:
   a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
   b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
   c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
   d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
   e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof;
ii. a cleavable linker; and
iii. a cytotoxic agent.

In some aspects of the pharmaceutical composition, the amount of ADC administered is about 25 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, about 300 mg, about 350 mg, about 400 mg, about 450 mg, about 500 mg, about 600 mg, about 750 mg, or about 900 mg.

In some aspects of the pharmaceutical composition, the cleavable linker and cytotoxic agent combined have the formula:

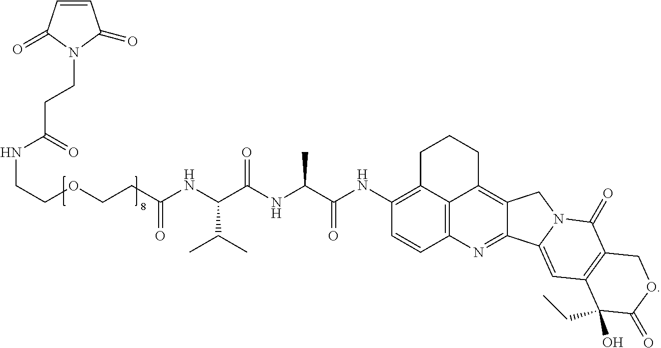

In some aspects of the pharmaceutical composition, the antibody or antigen binding fragment of the ADC comprises a VH chain and a VL chain comprising the amino acid sequence of SEQ ID NO: 45 and SEQ ID NO: 34, respectively, or a functional variant thereof.

The present disclosure further provides a kit comprising a pharmaceutical composition as described above and elsewhere herein. In some aspects, the kit further comprises instructions for administering the pharmaceutical composition.

The present disclosure further provides a pharmaceutical composition as described above and elsewhere herein comprising an ADC that specifically binds to B7-H4, for use in treating cancer. The treatment may be in accordance with any of the methods of treating cancer using an ADC that specifically binds to B7-H4 as described herein. In a related aspect, the present disclosure further provides use of a pharmaceutical composition as described above and elsewhere herein comprising an ADC that specifically binds to B7-H4, in the manufacture of a medicament for treating cancer. The treatment may be in accordance with any of the methods of treating cancer using an ADC that specifically binds to B7-H4 as described herein. In some aspects, the cancer comprises a cancer cell which expresses B7-H4.

In some aspects, the cancer is selected from endometrial cancer, ovarian cancer, biliary tract cancer, breast cancer, pancreatic cancer, prostate cancer, hematological cancer, cholangiocarcinoma, NSCLC (squamous and/or adenocarcinoma), gastrointestinal cancer such as gastric cancer and colorectal cancer, and lung cancer. In some aspects, the cancer is selected from endometrial cancer, ovarian cancer, breast cancer, pancreatic cancer, prostate cancer, hematological cancer, cholangiocarcinoma, NSCLC (squamous and/or adenocarcinoma), gastrointestinal cancer such as gastric cancer and colorectal cancer, and lung cancer. In some aspects, the cancer is a breast cancer selected from hormone receptor-positive (HR+) breast cancer, human epidermal growth factor receptor 2 positive (HER2+) breast cancer, and triple negative breast cancer (TNBC). In some aspects, the cancer is homologous recombination deficient (HRD) cancer.

In some aspects, the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L. In some aspects, the mutation in an HRD gene is selected from BRCA1, BRCA2, and ATM.

In some aspects, the disclosure provides a method of treating cancer in a subject, comprising administering to the subject an antibody-drug conjugate (ADC) that specifically binds to B7-H4, the ADC comprising:
i. an antibody or antigen binding fragment thereof which binds to a B7-H4 polypeptide, comprising:
   a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
   b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
   c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
   d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
   e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof;
ii. a cleavable linker; and
iii. a cytotoxic agent;
wherein at least about 25% of cancer cells in the subject are B7-H4 positive cells.

In some aspects, the B7-H4 positive cells are determined by measuring membranous B7-H4 expression prior to administration of the ADC, optionally wherein the B7-H4 expression is determined using immunohistochemistry (IHC), further wherein the IHC is optionally performed with an antibody reagent. The assessment may be based on measuring B7-H4 expression of a sample (e.g. a tissue sample, optionally a cancer sample) obtained from the subject. Thus, in some aspects, the disclosure is directed to a method for diagnosing a cancer sample as B7-H4 positive, comprising: assaying the cancer sample, optionally using immunohistochemistry (IHC), to determine whether at least 25% of the cells express membranous B7-H4, further wherein optionally the IHC is performed with an antibody reagent. If the cancer sample is diagnosed as B7-H4 positive, the subject may then be treated with an ADC that specifically binds to B7-H4 as described herein.

In some aspects, the ADC is administered in an amount from about 0.8 mg/kg to about 4.8 mg/kg. In some aspects, the ADC is administered to the subject once every three weeks. In some aspects, the subject has been previously treated with another chemotherapeutic agent. In some aspects, the subject has been previously treated with a platinum-based chemotherapeutic agent. In some aspects, the cancer is selected from endometrial cancer, ovarian cancer, biliary tract cancer, breast cancer, pancreatic cancer, prostate cancer, hematological cancer, cholangiocarcinoma, NSCLC (squamous and/or adenocarcinoma), gastrointestinal cancer such as gastric cancer and colorectal cancer, and lung cancer. In some aspects, the cancer is selected from endometrial cancer, ovarian cancer, breast cancer, pancreatic cancer, prostate cancer, hematological cancer, cholangiocarcinoma, NSCLC (squamous and/or adenocarcinoma), gastrointestinal cancer such as gastric cancer and colorectal cancer, and lung cancer In some aspects, the cancer is a breast cancer selected from hormone receptor-positive (HR+) breast cancer, human epidermal growth factor receptor 2 positive (HER2+) breast cancer, and triple negative breast cancer (TNBC). In some aspects, the cancer is homologous recombination deficient (HRD) cancer. In some aspects, the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L. In some aspects, the mutation in an HRD gene is selected from BRCA1, BRCA2, and ATM. In some aspects, the antibody or antigen binding fragment thereof comprises a VH chain and a VL chain comprising the amino acid sequence of SEQ ID NO: 45 and SEQ ID NO: 34, respectively, or a functional variant thereof. In some aspects, the antibody or antigen binding fragment thereof comprises a heavy chain comprising the amino acid sequence of SEQ ID NO: 51; and a light chain comprising the amino acid sequence of SEQ ID NO: 44. In some aspects, the antibody or antigen binding fragment thereof is a monoclonal antibody. In some aspects, the antibody or antigen binding fragment thereof is a humanized monoclonal antibody. In some aspects, the cleavable linker is an mp-PEG8-val-ala linker. In some aspects, the cytotoxic agent is a topoisomerase inhibitor. In some aspects, the topoisomerase inhibitor is a compound of Formula A*:

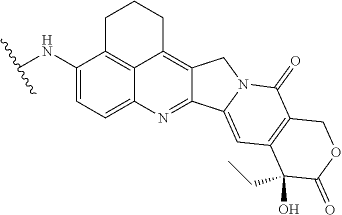 [SMILES: *Nc1ccc2nc3c(c4c2c1CCC4)Cn1c-3cc2c(c1=O)COC(=O)[C@]2(O)CC]

In some aspects, the ii) cleavable linker and iii) cytotoxic agent together are selected from the following compounds:

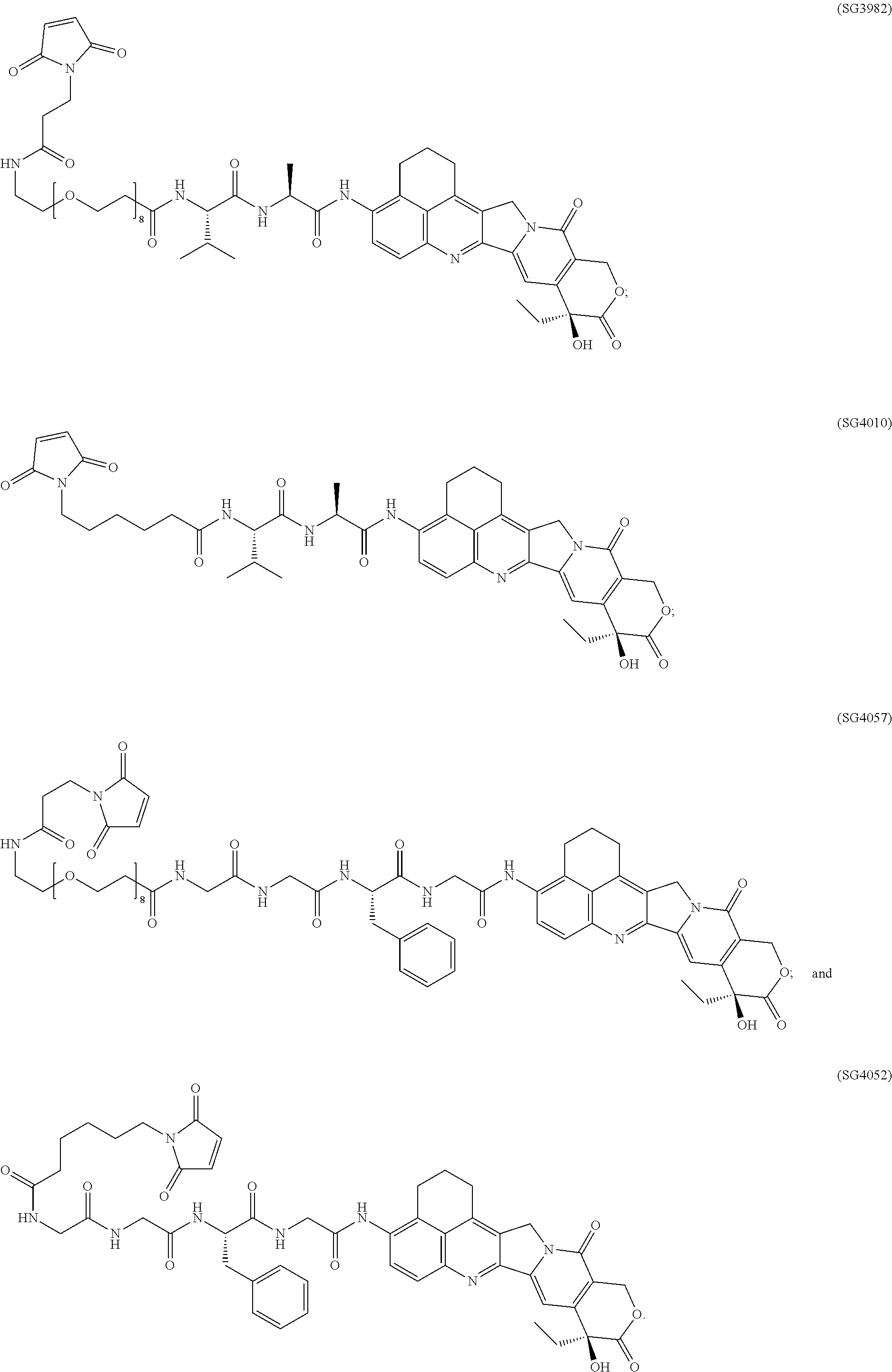

In some aspects, the ii) linker and iii) cytotoxic agent together are the compound SG3932. In some aspects, the ADC has a drug to antibody ratio (DAR) of between about 1 and about 8. In some aspects, the ADC has a DAR of about 8.

### BRIEF DESCRIPTION OF THE DRAWINGS

The following drawings form part of the present specification and are included to further demonstrate exemplary aspects of the present disclosure.

FIG. 1 is a schematic diagram showing the master protocol structure of treatment as described in the Examples. The master protocol structure comprises sub-study 1 for AZD8205 monotherapy, and one or more combinational sub-studies using AZD8205 with another active pharmaceutical agent.

FIG. 2 is a schematic diagram showing an example method of treatment comprising a monotherapy dose escalation phase (Part A) and a monotherapy dose expansion phase (Part B) as described in the Examples.

FIG. 3 is a chart of human ovarian carcinoma (OVCA) subjects' responses to AZD8205 treatment at an amount of 0.8 mg/kg, 1.6 mg/kg, 2.4 mg/kg, and 3.2 mg/kg, respectively. The responses are confirmed by detecting cancer antigen 125 (CA-125) levels in the subjects. Subjects with measurable CA-125 (>2× upper limit of normal (ULN)) are shown. Circulating tumor DNA (ctDNA) is used for detecting molecular response (MR), where P-MR indicates a partial molecular response (≥50% reduction of ctDNA from the base), C-MR indicates a complete molecular response (clearance), N-MR indicates no molecular response (<50% reduction, no change, or increase of ctDNA).

FIG. 4 shows clinical AZD8205 activity observed in Breast Cancer (BC) and Ovarian Cancer (OvCA), with preliminary signs of activity in Endometrial Cancer (EC) per Example 8.

FIG. 5 shows plots of AZD8205 efficiency as measured by the percent change in tumor growth versus the percentage of cells expressing B7-H4 in mouse xenograft studies as described in Example 9.

FIG. 6 is a plot of the Youden statistic versus the percentage of cells expressing B7-H4 for the mouse xenograft studies as described in Example 9. As can be seen in FIG. 6, the Youden statistic begins to plateau at 25% B7-H4.

FIG. 7 shows objective response rates (ORRs) to AZD8205 for Heavily Pre-Treated Subjects per Example 10.

### DETAILED DESCRIPTION

### Definitions

Unless otherwise defined herein, scientific, and technical terms used in the present disclosure shall have the meanings that are commonly understood by one of ordinary skill in the art. Further, unless otherwise required by context, singular terms shall include pluralities and plural terms shall include the singular.

As used herein, “a” or “an” may mean one or more. As used herein, when used in conjunction with the word “comprising,” the words “a” or “an” may mean one or more than one. As used herein, “another” or “a further” may mean at least a second or more.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited elements or method steps.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% or higher variability (either “greater than” or “less than” the indicated value), depending on the situation. In some aspects, one of skill in the art will understand the level of variability indicated by the term “about,” due to the context in which it is used herein. It should also be understood that use of the term “about” also includes the specifically recited value.

The use of the term “for example” and its corresponding abbreviation “e.g.,” (whether italicized or not) means that the specific terms recited are representative examples and aspects of the disclosure that are not intended to be limited to the specific examples referenced or cited unless explicitly stated otherwise.

Ranges provided herein, of any type, include all values within a particular range described and values about an endpoint for a particular range. As used herein, “between” is a range inclusive of the ends of the range. For example, a number between x and y explicitly includes the numbers x and y, and any numbers that fall within x and y.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxyl-terminal portion of each chain typically defines a constant domain responsible for effector function. Thus, in a naturally occurring antibody, a full-length heavy chain immunoglobulin polypeptide includes a variable domain (VH) and three constant domains (C_{H1}, C_{H2}, and C_{H3}) and a hinge region between C_{H1} and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus. Those of skill in the art, however, would appreciate that the locations of the domains in a naturally occurring antibody can be modified in certain antibody-like binding protein formats without a loss of antigen-binding capability. Classes of human light chains are termed kappa and lambda light chains.

In some aspects, the light chain constant region is a kappa chain. In some aspects, light chain constant region is a lambda chain.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4. A heavy chain complementarity determining region may be referred to as a “HCDR” or a “CHDR” herein. Similarly, a light chain complementarity determining region may be referred to as a “LCDR” or a “CLDR” herein.

The term “antibody fragment” refers to a portion of an intact or full-length chain or an antibody, generally the target binding or variable region. Examples of antibody fragments include, but are not limited to, F_{ab}, F_{ab′}, F_{(ab′)2} and Fᵥ fragments. As used herein, the term “functional fragment” is generally synonymous with “antibody fragment,” and with respect to antibodies, can refer to antibody fragments such as Fᵥ, F_{ab}, F_{(ab′)2}.

Reference to the numbering of amino acid residues described herein is performed according to the EU numbering system (also described in Kabat et al, Sequences of Proteins of Immunological Interest, 5th Ed. Public Health Service, National Institutes of Health, Bethesda, MD. (1991)).

The term “human antibody,” as used herein, includes antibodies having variable and constant regions substantially corresponding to human germline immunoglobulin sequences. In some aspects, human antibodies are produced in non-human mammals, including, but not limited to, rodents, such as mice and rats, and lagomorphs, such as rabbits. In other aspects, human antibodies are produced in hybridoma cells. In still other aspects, human antibodies are produced recombinantly. In some aspects, the bispecific binding protein is a human or humanized antibody.

The term “antigen” or “target antigen” as used herein refers to a molecule or a portion of a molecule that is capable of being recognized by and bound by binding proteins of the disclosure. The target antigen is capable of being used in an animal to produce antibodies capable of binding to an epitope of that antigen. A target antigen may have one or more epitopes.

The term “epitope” as used herein refers to a region or structural element of an antigen that is recognized and bound by a binding protein of the disclosure. More precisely, the epitope is the specific structure that is bound by the CDRs of the binding protein. Epitopes can comprise protein structural elements, carbohydrates or even portions of lipid structures found in membranes. A binding protein is said to specifically bind an antigen when it preferentially recognizes its antigen target in a complex mixture of proteins and/or macromolecules. The term “specifically binds” refers to a binding protein that specifically binds to a molecule or a fragment thereof (e.g., antigen). A binding protein that specifically binds a molecule or a fragment thereof may bind to other molecules with lower affinity as determined by, for example, immunoassays, BIAcore, or other assays known in the art. In particular, antibodies or fragments that specifically bind to at least one molecule or a fragment thereof can compete off molecules that bind non-specifically. The present disclosure specifically encompasses antibodies with multiple specificities (e.g., an antibody with specificity for two or more discrete antigens. For example, a bispecific antibody can bind to two adjacent epitopes on a single target antigen, or can bind to two different antigens.

The term “native Fc” as used herein refers to a molecule comprising the sequence of a non-antigen binding fragment resulting from digestion of an antibody or produced by other means, whether in monomeric or multimeric form, and can contain the hinge region. The original immunoglobulin source of the native Fc is preferably of human origin and can be any of the immunoglobulins. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a native Fc is a disulfide-bonded dimer resulting from papain digestion of an IgG. The term “native Fc” as used herein is generic to the monomeric, dimeric, and multimeric forms.

The term “Fc variant” as used herein refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. Furthermore, a native Fc comprises regions that can be removed or mutated to produce an Fc variant to alter certain residues that provide structural features or biological activity that are not required for the binding proteins of the disclosure. Thus, the term “Fc variant” comprises a molecule or sequence that lacks one or more native Fc sites or residues, or in which one or more Fc sites or residues has been modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The term “Fc domain” as used herein encompasses native Fc and Fc variants and sequences as defined above. As with Fc variants and native Fc molecules, the term “Fc domain” includes molecules in monomeric or multimeric form, whether digested from whole antibody or produced by other means.

The term “treating” or “treatment” refers to administering a compound or pharmaceutical composition to a subject in order to effect an alteration or improvement of a disease, disorder, or condition in the subject. The terms “treatment” or “treat” as used herein may refer to both therapeutic treatment and prophylactic or preventative measures. Those in need of treatment include subjects having a disease or condition as well as those prone to having disease or condition or those for which a disease or condition is to be prevented.

The term “dose” means a specified quantity of a compound or pharmaceutical agent provided in a single administration, or in a specified time period. In some aspects, a dose can be administered in two or more boluses, tablets, or injections. For example, in some aspects, where subcutaneous administration is desired, the desired dose may require a volume not easily accommodated by a single injection. In such aspects, two or more injections can be used to achieve the desired dose. In some aspects, a dose can be administered in two or more injections to minimize injection site reaction in an individual. In other aspects, the compound or pharmaceutical agent is administered by infusion over an extended period of time or continuously. Doses can be stated as the amount of pharmaceutical agent per hour, day, week or month.

The terms “subject”, “individual” and “patient” are used interchangeably herein to refer to a mammalian subject. In one aspect the “subject” is a human, domestic animals, farm animals, sports animals, and zoo animals, e.g., humans, non-human primates, dogs, cats, guinea pigs, rabbits, rats, mice, horses, cattle, etc. In one aspect, the subject is a cynomolgus monkey (Macaca fascicularis). In a preferable aspect, the subject is a human. In methods of the invention, the subject may not have been previously diagnosed as having cancer. Alternatively, the subject may have been previously diagnosed as having cancer. The subject may also be one who exhibits disease risk factors, or one who is asymptomatic for cancer. The subject may also be one who is suffering from or is at risk of developing cancer. Thus, in one aspect, a method of the invention can be used to confirm the presence of cancer in a subject. For example, the subject may previously have been diagnosed with cancer by alternative means. In one aspect, the subject has been previously administered a cancer therapy.

The term “efficacy” means the ability to produce a desired effect. A “therapeutically effective dose” or “therapeutic dose” is an amount sufficient to effect desired clinical results (i.e., achieve therapeutic efficacy). A therapeutically effective dose can be administered in one or more administrations.

The term “side effects” means physiological disease and/or conditions attributable to a treatment other than the desired effects. In some aspects, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality. A “disease” or “condition” refers to any condition that would benefit from treatment using the methods of the disclosure. “Disease” and “condition” are used interchangeably herein and include chronic and acute disorders or diseases, including those pathological conditions that predispose a patient to the disorder in question. In some aspects, the disease is a tumor. In some aspects, the disease is a solid tumor. In some aspects, the disease is cancer. In some aspects, the cancer is one or more of endometrial cancer, ovarian cancer, breast cancer, biliary tract cancer, colorectal cancer, prostate cancer, cervical cancer, uterine cancer, testicular cancer, bladder cancer, head and neck cancer, melanoma, pancreatic cancer, renal cell carcinoma, and lung cancer.

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

The terms “pharmaceutical composition” or “therapeutic composition” as used herein refer to a compound or composition capable of inducing a desired therapeutic effect when properly administered to a subject. In some aspects, the disclosure provides a pharmaceutical composition comprising a pharmaceutically acceptable carrier and a therapeutically effective amount of binding proteins of the disclosure.

The terms “pharmaceutically acceptable carrier” or “physiologically acceptable carrier” as used herein refer to one or more formulation materials suitable for accomplishing or enhancing the delivery of one or more binding proteins of the disclosure.

### Methods of Treating Cancer

The disclosure relates to a method of treating a cancer in a subject comprising administering to the subject an antibody-conjugate (ADC) that specifically binds to B7-H4 in an amount from about 0.8 mg/kg to about 4.8 mg/kg. In some aspects, the administered amount of the ADC is from about 25 mg to about 900 mg per subject. The disclosure also provides compositions, including pharmaceutical compositions, and kits, comprising such ADC.

In some aspects, the ADC comprises:
i. an antibody or antigen binding fragment thereof which binds to a B7-H4 polypeptide, comprising:
   a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
   b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
   c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
   (d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
   (e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof;
ii. a cleavable linker; and
iii. a cytotoxic agent.

B7-H4 (also known as V-set domain-containing T-cell activation inhibitor 1, encoded by the VTCN1 gene) is a transmembrane polypeptide of the B7 family of co-stimulatory proteins. B7-H4 is understood to be expressed on the surface of antigen-presenting cells for interactions with ligands of immune cells (e.g., T-lymphocytes, with CD28 being a potential ligand). B7-H4 has been observed to be highly expressed on cells of various cancer types and is thought to be a tumor-associated antigen. Furthermore, B7-H4 expression is not limited to a particular cancer type, such that it represents a target antigen for treating a broad spectrum of cancer types.

A “B7-H4 polypeptide” may comprise the full length polypeptide sequence of B7-H4 (e.g., SEQ ID NO.: 55), or may comprise a fragment of B7-H4 of any length of the full length polypeptide sequence of B7-H4 (e.g., comprising a polypeptide sequence of 5%, 15%, 25%, 35%, 45%, 55%, 65%, 75%, 85% or 95% of the full length polypeptide sequence of B7-H4) which comprises an epitope which can bind (e.g. be bound by) an antibody or antigen binding fragment of the disclosure. The B7-H4 polypeptide may comprise a sequence having 75%, 80%, 85%, 90% or 90% sequence identity to the sequence of SEQ ID NO.: 55. Preferably, the B7-H4 polypeptide comprises the sequence of SEQ ID NO.: 55.

In some aspects, the antibody or antigen binding fragment thereof can bind to B7-H4 molecules across species, e.g., the antibody or fragment can bind to mouse B7-H4, rat B7-H4, rabbit, human B7-H4 and/or cynomolgus monkey B7-H4. In some aspects, the antibody or fragment can bind to human B7-H4 and cynomolgus monkey B7-H4. In some aspects, the antibody or antigen binding fragment can also bind to mouse B7-H4.

In some aspects, the antibody or antigen binding fragment thereof can specifically bind to B7-H4, e.g., human B7-H4 and cynomolgus monkey B7-H4, but does not specifically bind to human B7-H1, B7-H2, and/or B7-H3.

In some aspects, the antibody or antigen binding fragment thereof of the ADC comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of: SEQ ID NO: 1, SEQ ID NO: 2, SEQ ID NO: 3, SEQ ID NO: 4, SEQ ID NO: 5, and SEQ ID NO: 6, respectively, or a functional variant thereof. An antibody or antigen binding fragment thereof comprising said sequences may be referred to as “ZY0EPQ-E02” or “EPQ-E02” herein.

In some aspects, the antibody or antigen binding fragment thereof comprises: a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof. An antibody or antigen binding fragment thereof comprising said sequences may be referred to as “ZY0EQD-E02” or “EQD-E02” herein.

In some aspects, the antibody or antigen binding fragment thereof of the ADC comprises a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof. An antibody or antigen binding fragment thereof comprising said sequences may be referred to as “ZY0EOB-F05” or “EOB-F05” herein.

In some aspects, the antibody or antigen binding fragment thereof of the ADC comprises a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof. An antibody or antigen binding fragment thereof comprising said sequences may be referred to as “ZY0E05-E07” or “E05-E07” herein.

In some aspects, the antibody or antigen binding fragment thereof of the ADC comprises a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof. An antibody or antigen binding fragment thereof comprising said sequences may be referred to as “ZY0EPO-C07” or “EPO-C07” herein.

In some aspects, the antibody or antigen binding fragment thereof having CDRs with the amino acid sequence disclosed above have beneficial advantages, such as high targeting specificity or binding activity toward B7-H4 peptide on the surface of cancer cells.

Additionally or alternatively, an antibody or antigen binding fragment thereof described herein may be described by means of a variable heavy chain (VH) and a variable light chain (VL) thereof.

In some aspects, the antibody or antigen binding fragment thereof comprises: a variable heavy (VH) chain and a variable light (VL) chain comprising the amino acid sequence of SEQ ID NO: 45 and SEQ ID NO: 34, respectively, or a functional variant thereof; a VH chain and a VL chain comprising the amino acid sequence of SEQ ID NO: 33 and SEQ ID NO: 34, respectively, or a functional variant thereof; a VH chain and a VL chain comprising the amino acid sequence of SEQ ID NO: 43 and SEQ ID NO: 34, respectively, or a functional variant thereof; a VH chain and a VL chain comprising the amino acid sequence of SEQ ID NO: 46 and SEQ ID NO: 34, respectively, or a functional variant thereof; a VH chain and a VL chain comprising the amino acid sequence of SEQ ID NO: 47 and SEQ ID NO: 34, respectively, or a functional variant thereof; a VH chain and a VL chain comprising the amino acid sequence of SEQ ID NO: 31, and SEQ ID NO: 32, respectively, or a functional variant thereof; a VH chain and a VL chain comprising the amino acid sequence of SEQ ID NO: 35 and SEQ ID NO: 36, respectively, or a functional variant thereof; a VH chain and a VL chain comprising the amino acid sequence of SEQ ID NO: 37 and SEQ ID NO: 38, respectively, or a functional variant thereof; or a VH chain and a VL chain comprising the amino acid sequence of SEQ ID NO: 39 and SEQ ID NO: 40, respectively, or a functional variant thereof.

For example, in one aspect, the antibody or antigen binding fragment thereof of the ADC comprises: (i) a variable heavy chain comprising an amino acid sequence having at least 70%, 75%, 80%, 90%, 95% or 100% sequence identity to the amino acid sequence of SEQ ID NO: 31, 33, 35, 37, 39, 43, 45, 46, or 47, or a functional variant thereof; and (ii) a variable light chain comprising an amino acid sequence having at least 70%, 75%, 80%, 90%, 95% or 100% sequence identity to the amino acid sequence of SEQ ID NO: 32, 34, 36, 38, or 40, or a functional variant thereof.

In some aspects, the antibody or antigen binding fragment thereof comprises: a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof.

In some aspects, the antibody or antigen binding fragment thereof comprises a VH chain and a VL chain comprising the amino acid sequence of SEQ ID NO: 45 and SEQ ID NO: 34, respectively, or a functional variant thereof. In some aspects, the antibody or antigen binding fragment thereof comprises a VH chain having an amino acid sequence that is at least 90, 91, 92, 93, 94, 95, 96, 97, 98, or 99% identical to the amino acid sequence of SEQ ID NO: 45. In some aspects, the antibody or antigen binding fragment thereof comprises a VL chain having an amino acid sequence that is at least 90, 91, 92, 93, 94, 95, 96, 97, 98, or 99% identical to the amino acid sequence of SEQ ID NO: 34.

In some aspects, the antibody or antigen-binding fragment thereof can include, in addition to a VH and a VL, and optionally a heavy chain constant region or fragment thereof, a light chain constant region or fragment thereof. In some aspects, the light chain constant region is a kappa lambda light chain constant region, e.g., a human kappa constant region or a human lambda constant region.

In some aspects, the antibody or antigen binding fragment thereof comprises a heavy chain constant region comprising the amino acid sequence of SEQ ID NO: 41. In some aspects, the antibody or antigen binding fragment thereof comprises a heavy chain constant region that is at least 90, 91, 92, 93, 94, 95, 96, 97, 98, or 99% identical to the amino acid sequence of SEQ ID NO:41.

In some aspects, the antibody or antigen binding fragment thereof comprises a heavy chain constant region comprising the amino acid sequence of SEQ ID NO: 52. In some aspects, the antibody or antigen binding fragment thereof comprises a heavy chain constant region that is at least 90, 91, 92, 93, 94, 95, 96, 97, 98, or 99% identical to the amino acid sequence of SEQ ID NO:52.

In some aspects, the antibody or antigen binding fragment thereof comprises a light chain constant region comprising the amino acid sequence of SEQ ID NO: 42. In some aspects, the antibody or antigen binding fragment thereof comprises a light chain constant region that is at least 90, 91, 92, 93, 94, 95, 96, 97, 98, or 99% identical to the amino acid sequence of SEQ ID NO:42.

In some aspects, the antibody or antigen binding fragment thereof comprises a heavy chain comprising the amino acid sequence of SEQ ID NO: 51; and a light chain comprising the amino acid sequence of SEQ ID NO: 44. In some aspects, the antibody or antigen binding fragment thereof comprises a heavy chain that is at least 90, 91, 92, 93, 94, 95, 96, 97, 98, or 99% identical to the amino acid sequence of SEQ ID NO:51. In some aspects, the antibody or antigen binding fragment thereof comprises a light chain that is at least 90, 91, 92, 93, 94, 95, 96, 97, 98, or 99% identical to the amino acid sequence of SEQ ID NO:44.

In some aspects, the antibody or antigen binding fragment thereof comprises a heavy chain comprising the amino acid sequence of SEQ ID NO: 48; and a light chain comprising the amino acid sequence of SEQ ID NO: 44. In some aspects, the antibody or antigen binding fragment thereof comprises a heavy chain that is at least 90, 91, 92, 93, 94, 95, 96, 97, 98, or 99% identical to the amino acid sequence of SEQ ID NO:48. In some aspects, the antibody or antigen binding fragment thereof comprises a light chain that is at least 90, 91, 92, 93, 94, 95, 96, 97, 98, or 99% identical to the amino acid sequence of SEQ ID NO:44.

In some aspects, the antibody or antigen binding fragment thereof is a monoclonal antibody. In some aspects, the antibody or antigen binding fragment thereof is a humanized monoclonal antibody.

Advantageously, the disclosure has demonstrated that an antibody or antigen binding fragment of described above may target a broader spectrum of B7-H4 expressing cells when compared with existing (commercially) available antibodies reported to target B7-H4. Thus, not only has the disclosure provided an antibody (or antigen binding fragment) thereof having affinity and specificity for a clinically relevant target, but has also demonstrated a unique advantage (e.g., unexpected technical effect) associated therewith.

In some aspects, the cancer is selected from ovarian cancer, biliary tract cancer, breast cancer, pancreatic cancer, prostate cancer, hematological cancer, endometrial cancer, cholangiocarcinoma, NSCLC (squamous and/or adenocarcinoma), gastrointestinal cancer such as gastric cancer and colorectal cancer, and lung cancer.

In some aspects, the cancer is endometrial cancer.

In some aspects, the cancer is ovarian cancer.

In some aspects, the cancer is biliary tract cancer.

In some aspects, the cancer is breast cancer.

In some aspects, the cancer is a breast cancer selected from hormone receptor-positive (HR+) breast cancer, human epidermal growth factor receptor 2 positive (HER2+) breast cancer, and triple negative breast cancer (TNBC).

In some aspects, the cancer is homologous recombination deficient (HRD) cancer.

In some aspects, the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L. In some aspects, the mutation in an HRD gene is selected from BRCA1, BRCA2, and ATM.

In some aspects, the antibody or antigen binding fragment thereof of the ADC binds an OVCAR4 cell line. In some aspects, an antibody or antigen binding fragment thereof described herein may bind to an OVCAR4 cell line and/or a CHO cell line (e.g., which may lack an exogenous nucleic acid encoding B7-H4). For example, the antibody or antigen binding fragment thereof binds to a B7-H4 (e.g., a B7-H4 epitope) of an OVCAR4 cell line and/or a CHO cell line (e.g., which may lack an exogenous nucleic acid encoding B7-H4).

In some aspects, the antibody or antigen binding fragment thereof binds to an OVCAR4 cell line and/or CHO cell line (e.g. which may lack an exogenous nucleic acid encoding B7-H4) with higher affinity when compared to one or more antibody selected from E Biosciences 14-5949 anti-Human B7H4 mouse IgG, US biological B0000-35B anti Human B7H4 mouse IgG, R and D systems AF2514 anti-Mouse B7H4 goat IgG1, Sigma SAB2500141 anti B7H4 Goat IgG1, Isotype 1 CAT004 SP06-003, Isotype 2 R and D Normal goat IgG control (AB-108C), AdD serotec MCA2632, Epitomics 2516-1, eBiosciences, 145972-82, eBioscience 145970-85, or a combination thereof. The affinity (e.g., binding affinity) can be measured by any suitable method of measuring binding affinity described herein.

The OVCAR4 cell line is a human ovary carcinoma cell line, the CHO cell line is an epithelial cell line derived from the ovary of the Chinese hamster, and are widely obtainable.

In some aspects, at least about 25% of the cancer cells are B7-H4 positive cells. In some aspects, at least about 5%, 10%, 15%, 20%, 25%, 30%, 35%, 40%, 45%, 50%, 55%, 60%, 65%, 70% or 75% of the cancer cells are B7-H4 positive cells. In some aspects, a B7-H4 positive cell is a cell that expresses B7-H4 on its cell surface. In some aspects, the percentage of B7-H4 positive cells is determined by analyzing a sample, e.g., a biopsy of cancer tissue from the subject. In some aspects, the percentage of B7-H4 positive cells is determined using an antibody for detection of B7-H4.

In some aspects, the percentage of cells in cancer that are B7-H4 positive is determined by immunohistochemistry (IHC). IHC is a laboratory method that uses antibodies to detect antigens (biomarkers) in a sample of tissue. In aspects, the antibodies are linked to an enzyme or a fluorescent dye. After the antibodies bind to the antigen in the tissue sample, the enzyme or dye is activated, and the antigen can then be detected using a microscope or other visualization device.

In some aspects, the antibody or antigen binding fragment thereof is linked to the cytotoxic agent by a linker. In some aspects, the antibody or antigen binding fragment thereof is conjugated to the cytotoxic agent by a linker. As used herein, “conjugated” means linked via a covalent or ionic bond. In some aspects, the linker is attached (e.g., conjugated) in a cleavable manner to an amino residue, for example, an amino acid of the antibody or antigen binding fragment of the ADC. In some aspects, the cleavable linker of the ADC is an mp-PEG8-val-ala linker.

The cytotoxic agent may be referred to herein as an “agent” or “active agent.” In some aspects, the cytotoxic agent is a drug. The cytotoxic agent or cytotoxin can be any molecule known in the art that inhibits or prevents the function of cells and/or causes destruction of cells (cell death), and/or exerts anti-neoplastic/anti-proliferative effects. A number of classes of cytotoxic agents are known to have potential utility in ADC molecules. These include, but are not limited to, topoisomerase I inhibitors, amanitins, auristatins, daunomycins, doxorubicins, duocarmycins, dolastatins, enediynes, lexitropsins, taxanes, puromycins, maytansinoids, vinca alkaloids, tubulysins and pyrrolobenzodiazepines (PBDs). Examples of such cytotoxic agents are AFP, MMAF, MMAE, AEB, AEVB, auristatin E, paclitaxel, docetaxel, CC-1065, SN-38, topotecan, morpholino-doxorubicin, rhizoxin, cyanomorpholino-doxorubicin, dolastatin-10, echinomycin, combretatstatin, chalicheamicin, maytansine, DM-1, vinblastine, methotrexate, and netropsin, and derivatives and analogs thereof.

In some aspects, the cytotoxic agent is a drug. In some aspects, the cytotoxic agent of the ADC is a topoisomerase inhibitor, tubulysin derivative, a pyrrolobenzodiazepine, or a combination thereof. In some aspects, the cytotoxic agent is one of the cytotoxic agents published in US Patent Application Publication Nos. US 2022-0211863, and US 2017-0291955, both of which are incorporated by reference herein in its entirety.

In preferred aspects, the cytotoxic agent is a topoisomerase inhibitor. Topoisomerase inhibitors are chemical compounds that block the action of topoisomerase (topoisomerase I and II), which is a type of enzyme that controls the changes in DNA structure by catalyzing the breaking and rejoining of the phosphodiester backbone of DNA strands during the normal cell cycle. The topoisomerase I inhibitor can be, for example, SG3932, SG4010, SG4057 or SG4052 provided below.

The present disclosure provides the cytotoxic agent (Dug Unit) of the ADC, which is connected to a Ligand Unit of the ADC via the linker (linking unit) described above. The Ligand Unit is preferably an antibody or antigen binding fragment thereof. In some aspects, the present disclosure provides a conjugate comprising the following topoisomerase inhibitor derivative (A*, the Drug Unit):

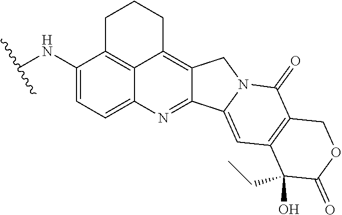 [SMILES: *Nc1ccc2nc3c(c4c2c1CCC4)Cn1c-3cc2c(c1=O)COC(=O)[C@]2(O)CC]

In some aspects, the cytotoxic agent is a topoisomerase inhibitor and wherein the cleavable linker and cytotoxic agent of the ADC together comprise a compound of formula I:

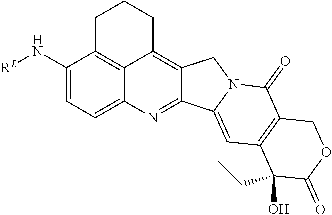

and salts and solvates thereof. R^{L} is the cleavable linker described above.

In some aspects, the linker and cytotoxic agent together comprise one of the following compounds:

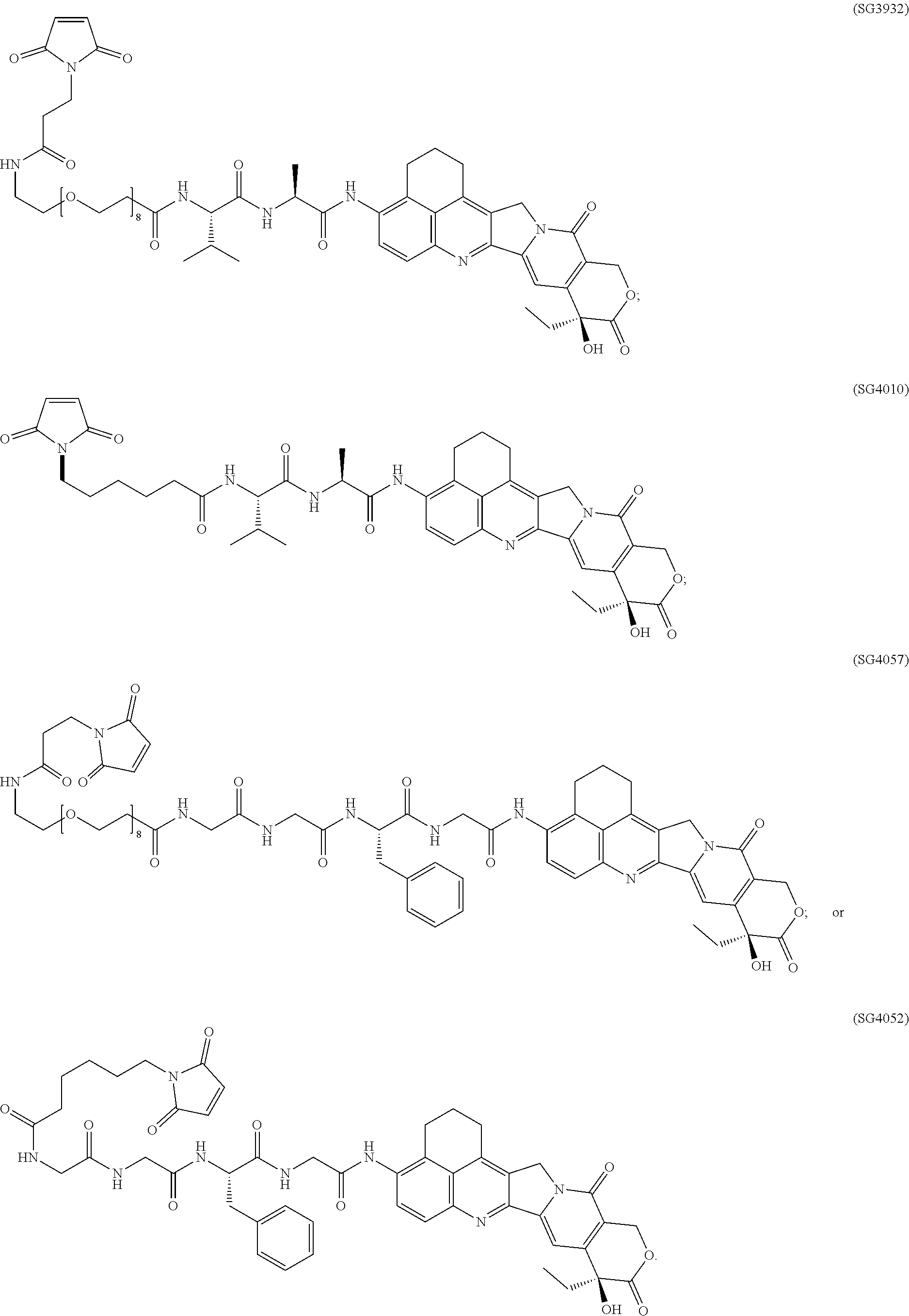

In some aspects, the linker and cytotoxic agent together are the compound SG3932.

The cytotoxic agent is typically linked to, or “loaded onto” the antibody or antigen-binding fragment. The agent loading (p) is the average number of agent(s) per antibody or antigen-binding fragment (e.g., the Ligand Unit).

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

Typically, fewer than the theoretical maximum of drug moieties are conjugated to an antibody during a conjugation reaction. An antibody may contain, for example, many lysine residues that do not react with the Drug Linker. Only the most reactive lysine groups may react with an amine-reactive linker reagent. Also, only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an ADC may be controlled in several different manners, including: (i) limiting the molar excess of Drug Linker relative to antibody, (ii) limiting the conjugation reaction time or temperature, and (iii) partial or limiting reductive conditions for cysteine thiol modification.

Certain antibodies have reducible interchain disulfides, i.e., cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody and which do not form intrachain or intermolecular disulfide linkages (Junutula, et al., 2008b Nature Biotech., 26(8):925-932; Dornan et al (2009) Blood 114(13):2721-2729; U.S. Pat. Nos. 7,521,541; 7,723,485; WO2009/052249). The engineered cysteine thiols may react with Drug Linkers of the present invention which have thiol-reactive, electrophilic groups such as maleimide or alpha-halo amides to form ADC with cysteine engineered antibodies. The location of the drug unit can thus be designed, controlled, and known. The drug loading can be controlled since the engineered cysteine thiol groups typically react with drug-linker reagents in high yield. Engineering an IgG antibody to introduce a cysteine amino acid by substitution at a single site on the heavy or light chain gives two new cysteines on the symmetrical antibody.

In some aspects, the average number of agents per antibody (or antigen-binding fragment thereof) is in the range 1 to 20. In some aspects the range is selected from 1 to 10, 2 to 10, 2 to 8, 2 to 6, and 4 to 10. In some aspects, there is one agent per antibody (or antigen-binding fragment thereof). In some aspects, the number of agents per antibody (or antigen-binding fragment thereof) can be expressed as a ratio of agent (i.e., drug) to antibody. This ratio is referred to as the Drug to Antibody Ratio (DAR). The DAR is the average number of drugs (i.e., agents) linked to each antibody. In some aspects of the present disclosure, the DAR is in the range 1 to 20. In some aspects the range of DAR is selected from 1 to 10, 2 to 10, 2 to 8, 2 to 6, and 4 to 10. In some aspects, the DAR is between about 1 and about 8. In a particular aspect of the present disclosure, the DAR is about 8. In a particular aspect of the present disclosure, the DAR is 8.

In some aspects, the ADC is AZD8205. AZD8205 is an anti-B7H4 Ab conjugated to topoisomerase inhibitor (TOPO) warhead. In particular, AZD8205 has a heavy chain with SEQ ID NO: 51 and a light chain with SEQ ID NO: 44, and is conjugated to SG3932. Typically, the DAR is about 8. In some aspects, AZD8205 is targeted to ovarian cancer and cholangiocarcinoma (CCA). In some aspects, AZD8205 is targeted to triple negative breast cancer (TNBC). In some aspects, AZD8205 is targeted to endometrial cancer.

In some aspects, the amount of ADC administered is about 0.5 mg/kg to about 8 mg/kg. In some aspects, the amount of ADC administered is about 0.8 mg/kg to about 4.8 mg/kg. In some aspects, the amount of ADC administered is about 1.2 mg/kg to about 3.6 mg/kg. In some aspects, the amount of ADC administered is about 1.4 mg/kg to about 3.2 mg/kg. In some aspects, the amount of ADC administered is about 1.6 mg/kg to about 2.4 mg/kg. In some aspects, the amount of ADC administered is about 1.8 mg/kg to about 2.2 mg/kg.

In some aspects, the amount of ADC administered is about 0.5 mg/kg, about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.8 mg/kg, about 3.0 mg/kg, about 3.2 mg/kg, about 3.6 mg/kg, about 4.0 mg/kg, about 4.4 mg/kg, about 4.8 mg/kg, about 5.5 mg/kg, about 6.0 mg/kg, about 7.0 mg/kg, or about 8 mg/kg.

In some aspects, the amount of ADC administered is about 1.2 mg/kg, about 1.6 mg/kg, about 1.8 mg/kg, about 2.0 mg/kg, about 2.2 mg/kg, about 2.4 mg/kg, or about 3.2 mg/kg.

In some aspects, the amount of ADC administered is about 1.6 mg/kg. In some aspects, the amount of ADC administered is about 2.0 mg/kg. In some aspects, the amount of ADC administered is about 2.4 mg/kg. In some aspects, the amount of ADC administered is about 3.2 mg/kg.

In some aspects, the amount of ADC administered is about 25 mg to about 900 mg. In some aspects, the amount of ADC administered is about 40 mg to about 600 mg. In some aspects, the amount of ADC administered is about 60 mg to about 450 mg. In some aspects, the amount of ADC administered is about 80 mg to about 300 mg. In some aspects, the amount of ADC administered is about 100 mg to about 240 mg. In some aspects, the amount of ADC administered is about 120 mg to about 200 mg. In some aspects, the amount of ADC administered is about 140 mg to about 180 mg.

In some aspects, the amount of ADC administered is about 25 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, about 300 mg, about 350 mg, about 400 mg, about 450 mg, about 500 mg, about 600 mg, about 750 mg, or about 900 mg.

In some aspects, the amount of ADC administered is about 80 mg, about 100 mg, about 120 mg, about 140 mg, about 160 mg, about 180 mg, about 200 mg, about 240 mg, about 280 mg, or about 320.

In some aspects, the amount of ADC administered is about 120 mg. In some aspects, the amount of ADC administered is about 160 mg. In some aspects, the amount of ADC administered is about 200 mg.

In some aspects, the ADC is administered to the subject once every three weeks (Q3W). In some aspects, the ADC is administered to the subject once weekly (Q1W), once every two weeks (Q2W), once every four weeks (Q4W), once every five weeks (Q5W) or once every six weeks (Q6W).

In some aspects, the present disclosure further provides a method of treating cancer in a subject, comprising administering to the subject an antibody-drug conjugate (ADC) that specifically binds to B7-H4 in an amount from about 0.8 mg/kg to about 4.8 mg/kg. In some aspects, the ADC comprises: i. an antibody or antigen binding fragment thereof which binds to a B7-H4 polypeptide, comprising: a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof; and ii.a cleavable linker and cytotoxic agent conjugated to the antibody or antigen binding fragment thereof having the formula:

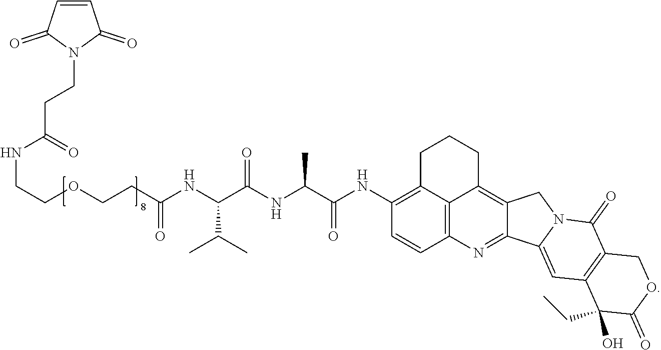

In some aspects, the antibody or antigen binding fragment thereof comprises a VH chain and a VL chain comprising the amino acid sequence of SEQ ID NO: 45 and SEQ ID NO: 34, respectively, or a functional variant thereof.

In some aspects, the cancer is selected from ovarian cancer, biliary tract cancer, breast cancer, pancreatic cancer, prostate cancer, hematological cancer, endometrial cancer, cholangiocarcinoma, NSCLC (squamous and/or adenocarcinoma), gastrointestinal cancer such as gastric cancer and colorectal cancer, and lung cancer.

In some aspects, the cancer is endometrial cancer.

In some aspects, the cancer is ovarian cancer.

In some aspects, the cancer is biliary tract cancer.

In some aspects, the cancer is breast cancer.

In some aspects, the cancer is a breast cancer selected from hormone receptor-positive (HR+) breast cancer, human epidermal growth factor receptor 2 positive (HER2+) breast cancer, and triple negative breast cancer (TNBC). In some aspects, the cancer is homologous recombination deficient (HRD) cancer.

In some aspects, the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L. In some aspects, the mutation in an HRD gene is selected from BRCA1, BRCA2, and ATM.

In some aspects, the amount of ADC administered is about 0.5 mg/kg to about 8 mg/kg. In some aspects, the amount of ADC administered is about 0.8 mg/kg to about 4.8 mg/kg. In some aspects, the amount of ADC administered is about 1.2 mg/kg to about 3.6 mg/kg. In some aspects, the amount of ADC administered is about 1.4 mg/kg to about 3.2 mg/kg. In some aspects, the amount of ADC administered is about 1.6 mg/kg to about 2.4 mg/kg. In some aspects, the amount of ADC administered is about 1.8 mg/kg to about 2.2 mg/kg.

In some aspects, the amount of ADC administered is about 0.5 mg/kg, about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.8 mg/kg, about 3.0 mg/kg, about 3.2 mg/kg, about 3.6 mg/kg, about 4.0 mg/kg, about 4.4 mg/kg, about 4.8 mg/kg, about 5.5 mg/kg, about 6.0 mg/kg, about 7.0 mg/kg, or about 8 mg/kg.

In some aspects, the amount of ADC administered is about 1.2 mg/kg, about 1.6 mg/kg, about 1.8 mg/kg, about 2.0 mg/kg, about 2.2 mg/kg, about 2.4 mg/kg, or about 3.2 mg/kg.

In some aspects, the amount of ADC administered is about 1.6 mg/kg. In some aspects, the amount of ADC administered is about 2.0 mg/kg. In some aspects, the amount of ADC administered is about 2.4 mg/kg. In some aspects, the amount of ADC administered is about 3.2 mg/kg.

In some aspects, the amount of ADC administered is about 25 mg to about 900 mg. In some aspects, the amount of ADC administered is about 40 mg to about 600 mg. In some aspects, the amount of ADC administered is about 60 mg to about 450 mg. In some aspects, the amount of ADC administered is about 80 mg to about 300 mg. In some aspects, the amount of ADC administered is about 100 mg to about 240 mg. In some aspects, the amount of ADC administered is about 120 mg to about 200 mg. In some aspects, the amount of ADC administered is about 140 mg to about 180 mg.

In some aspects, the amount of ADC administered is about 25 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, about 300 mg, about 350 mg, about 400 mg, about 450 mg, about 500 mg, about 600 mg, about 750 mg, or about 900 mg.

In some aspects, the amount of ADC administered is about 80 mg, about 100 mg, about 120 mg, about 140 mg, about 160 mg, about 180 mg, about 200 mg, about 240 mg, about 280 mg, or about 320.

In some aspects, the amount of ADC administered is about 120 mg. In some aspects, the amount of ADC administered is about 160 mg. In some aspects, the amount of ADC administered is about 200 mg.

In some aspects, the ADC is administered to the subject once every three weeks (Q3W). In some aspects, the ADC is administered to the subject once weekly (Q1W), once every two weeks (Q2W), once every four weeks (Q4W), once every five weeks (Q5W) or once every six weeks (Q6W).

In some aspects, the present disclosure further provides a method of treating cancer in a subject, comprising administering to the subject an antibody-drug conjugate (ADC) that specifically binds to B7-H4, the ADC comprising:
i. an antibody or antigen binding fragment thereof which binds to a B7-H4 polypeptide, comprising:
   f) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
   g) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
   h) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
   i) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
   j) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof;
ii. a cleavable linker; and
iii. a cytotoxic agent;
wherein at least about 25% of cancer cells in the subject are B7-H4 positive cells.

Cleavable linkers and cytotoxic agents of the ADC for use in this method are described herein. Cancers treatable by this method are described herein.

In some aspects, at least about 5%, 10%, 15%, 20%, 25%, 30%, 35%, 40%, 45%, 50%, 55%, 60%, 65%, 70% or 75% of the cancer cell are B7-H4 positive cells. In some aspects, a B7-H4 positive cell is a cell that expresses B7-H4 on its cell surface. In some aspects, the percentage of B7-H4 positive cells is determined by analyzing a sample, e.g., a biopsy of cancer tissue from the subject. In some aspects, the percentage of B7-H4 positive cells is determined using an antibody for detection of B7-H4.

In aspects of the method, the ADC is administered in an amount from about 0.8 mg/kg to about 4.8 mg/kg. In aspects, the amount of ADC administered is about 0.8 mg/kg, about 1.6 mg/kg, about 2.4 mg/kg, about 3.2 mg/kg, about 3.6 mg/kg or about 4.8 mg/kg. In aspects, the amount of ADC administered is about 1.6 mg/kg. In aspects, the amount of ADC administered is about 2.4 mg/kg. In aspects, the amount of ADC administered is about 3.2 mg/kg.

In other aspects of the method, the ADC is administered in an amount as disclosed herein.

In some aspects, the ADC is administered to the subject once every three weeks (Q3W). In some aspects, the ADC is administered to the subject once weekly (Q1W), once every two weeks (Q2W), once every four weeks (Q4W), once every five weeks (Q5W) or once every six weeks (Q6W).

In aspects of any of the methods or uses described herein, the subject has been previously treated with another chemotherapeutic agent. In some aspects, the subject has been previously treated with a platinum-based chemotherapeutic agent. In some aspects, the platinum-based chemotherapeutic agent is cisplatin, carboplatin, oxaliplatin, nedaplatin, lobaplatin or heptaplatin.

### Pharmaceutical Compositions

In some aspects, the present disclosure further provides a pharmaceutical composition comprising an antibody-drug conjugate (ADC) that specifically binds to B7-H4 which is administered in an amount from about 0.8 mg/kg to about 4.8 mg/kg. In some aspects, the ADC comprising: i. an antibody or antigen binding fragment thereof which binds to a B7-H4 polypeptide, comprising: a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof; and ii. a cleavable linker and cytotoxic agent conjugated to the antibody or antigen binding fragment thereof having the formula:

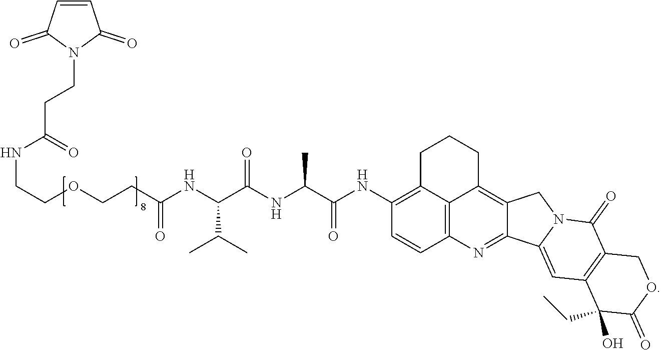

In some aspects, the amount of ADC administered is about 0.5 mg/kg to about 8 mg/kg. In some aspects, the amount of ADC administered is about 0.8 mg/kg to about 4.8 mg/kg. In some aspects, the amount of ADC administered is about 1.2 mg/kg to about 3.6 mg/kg. In some aspects, the amount of ADC administered is about 1.4 mg/kg to about 3.2 mg/kg. In some aspects, the amount of ADC administered is about 1.6 mg/kg to about 2.4 mg/kg. In some aspects, the amount of ADC administered is about 1.8 mg/kg to about 2.2 mg/kg.

In some aspects, the amount of ADC administered is about 0.5 mg/kg, about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.8 mg/kg, about 3.0 mg/kg, about 3.2 mg/kg, about 3.6 mg/kg, about 4.0 mg/kg, about 4.4 mg/kg, about 4.8 mg/kg, about 5.5 mg/kg, about 6.0 mg/kg, about 7.0 mg/kg, or about 8 mg/kg.

In some aspects, the amount of ADC administered is about 1.2 mg/kg, about 1.6 mg/kg, about 1.8 mg/kg, about 2.0 mg/kg, about 2.2 mg/kg, about 2.4 mg/kg, or about 3.2 mg/kg.

In some aspects, the amount of ADC administered is about 1.6 mg/kg. In some aspects, the amount of ADC administered is about 2.0 mg/kg. In some aspects, the amount of ADC administered is about 2.4 mg/kg. In some aspects, the amount of ADC administered is about 3.2 mg/kg.

In some aspects, the amount of ADC administered is about 25 mg to about 900 mg. In some aspects, the amount of ADC administered is about 40 mg to about 600 mg. In some aspects, the amount of ADC administered is about 60 mg to about 450 mg. In some aspects, the amount of ADC administered is about 80 mg to about 300 mg. In some aspects, the amount of ADC administered is about 100 mg to about 240 mg. In some aspects, the amount of ADC administered is about 120 mg to about 200 mg. In some aspects, the amount of ADC administered is about 140 mg to about 180 mg.

In some aspects, the amount of ADC administered is about 25 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, about 300 mg, about 350 mg, about 400 mg, about 450 mg, about 500 mg, about 600 mg, about 750 mg, or about 900 mg.

In some aspects, the amount of ADC administered is about 80 mg, about 100 mg, about 120 mg, about 140 mg, about 160 mg, about 180 mg, about 200 mg, about 240 mg, about 280 mg, or about 320.

In some aspects, the amount of ADC administered is about 120 mg. In some aspects, the amount of ADC administered is about 160 mg. In some aspects, the amount of ADC administered is about 200 mg.

In some aspects of a use of a pharmaceutical composition disclosed herein for treatment of cancer, the ADC is administered to the subject once every three weeks (Q3W). In some aspects of a use of a pharmaceutical composition disclosed herein for treatment of cancer, the ADC is administered to the subject once weekly (Q1W), once every two weeks (Q2W), once every four weeks (Q4W), once every five weeks (Q5W) or once every six weeks (Q6W).

In some aspects of a use of a pharmaceutical composition disclosed herein for treatment of cancer, a subject having cancer has been previously treated with another chemotherapeutic agent. In some aspects, the subject has been previously treated with a platinum-based chemotherapeutic agent. In some aspects, the platinum-based chemotherapeutic agent is cisplatin, carboplatin, oxaliplatin, nedaplatin, lobaplatin or heptaplatin.

In some aspects, the antibody or antigen binding fragment of the ADC comprises a VH chain and a VL chain comprising the amino acid sequence of SEQ ID NO: 45 and SEQ ID NO: 34, respectively, or a functional variant thereof. In some aspects, the antibody or antigen binding fragment of the ADC comprises a VH chain comprising the amino acid sequence that is at least 90% identical to the amino acid sequence of SEQ ID NO: 45 and a VL chain comprising the amino acid sequence that is at least 90% identical to the amino acid sequence of SEQ ID NO: 34.

In some aspects, the pharmaceutical compositions disclosed herein can be formulated with a pharmaceutically acceptable carrier, excipient, or stabilizer. In certain aspects, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which can be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

The present disclosure further provides a pharmaceutical composition as described above, for use in treating cancer. In some aspects, the cancer comprises a cancer cell which expresses B7-H4. In some aspects, the cancer is selected from ovarian cancer, biliary tract cancer, breast cancer, pancreatic cancer, prostate cancer, hematological cancer, endometrial cancer, cholangiocarcinoma, NSCLC (squamous and/or adenocarcinoma), gastrointestinal cancer such as gastric cancer and colorectal cancer, and lung cancer.

In some aspects, the cancer is biliary tract cancer.

In some aspects, the cancer is endometrial cancer.

In some aspects, the cancer is ovarian cancer.

In some aspects, the cancer is breast cancer. In some aspects, the cancer is a breast cancer selected from hormone receptor-positive (HR+) breast cancer, human epidermal growth factor receptor 2 positive (HER2+) breast cancer, and triple negative breast cancer (TNBC). In some aspects, the cancer is homologous recombination deficient (HRD) cancer. In some aspects, the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L. In some aspects, the mutation in an HRD gene is selected from BRCA1, BRCA2, and ATM.

### Kits

In some aspects, the present disclosure further provides a kit comprising any of the pharmaceutical compositions described above. In some aspects, the kit comprises instructions for administering the pharmaceutical composition. In some aspects, the kit comprises an additional anti-cancer agent as described herein.

### Antibody or Antigen Binding Fragment Sequences of the ADC

Aspects of the disclosure include antibody or antigen binding fragment in DuetMab format that bind B7-H4 polypeptide created using the sequences in Table 1 below. CDRs in Table 1 are determined based on the system in Kabat.

TABLE 1
Sequences of antibody or antigen binding fragments of the ADC

| | SEQ ID | |
|---|---|---|
| Identifier | NO | Sequence |
| ZYOEPQ-E02; CHDR1 | 1 | GYYWN |
| ZYOEPQ-E02; CHDR2 | 2 | EINHSGSTNYNPSLKS |
| ZYOEPQ-E02; CHDR3 | 3 | NLYNWNLDS |
| ZYOEPQ-E02; CLDR1 | 4 | RASQGIRNDLG |
| ZYOEPQ-E02; CLDR2 | 5 | VASSLQS |
| ZYOEPQ-E02; CLDR3 | 6 | LQHNSYPRT |
| ZYOEQD-E02; CHDR1 | 7 | GYYWN |
| ZYOEQD-E02; CHDR2 | 8 | EINHSGSTSYNPSLKS |
| ZYOEQD-E02; CHDR3 | 9 | VLYNWNVDS |
| ZYOEQD-E02; CLDR1 | 10 | RASQDIRNDVG |
| ZYOEQD-E02; CLDR2 | 11 | AASRLQS |
| ZYOEQD-E02; CLDR3 | 12 | LQHNSYPRT |
| ZYOEOB-F05; CHDR1 | 13 | SGGYYWS |
| ZYOEOB-F05; CHDR2 | 14 | NIYYSGSTYYNPSLKS |
| ZYOEOB-F05; CHDR3 | 15 | EKALATVTPSGYENYYTVDV |
| ZYOEOB-F05; CLDR1 | 16 | WASQGISSYLA |
| ZYOEOB-F05; CLDR2 | 17 | AASTLQS |
| ZYOEOB-F05; CLDR3 | 18 | QHLNSYPLT |
| ZY0E05-E07; CHDR1 | 19 | SGGYYWS |
| ZY0E05-E07; CHDR2 | 20 | NIYYSGSTYYNPSLKS |
| ZY0E05-E07; CHDR3 | 21 | EKALASVIPSGYENYYVVDV |
| ZY0E05-E07; CLDR1 | 22 | WASQGIAGYLA |
| ZY0E05-E07; CLDR2 | 23 | AASTLQS |
| ZY0E05-E07; CLDR3 | 24 | QHLNSYPLT |
| ZYOEPO-C07; CHDR1 | 25 | DYYMS |
| ZYOEP0-C07; CHDR2 | 26 | YISSSGSTIYYTDSVKG |
| ZYOEPO-C07; CHDR3 | 27 | DGVGFDY |
| ZYOEPO-C07; CLDR1 | 28 | RASQSVSSSYLA |
| ZYOEP0-C07; CLDR2 | 29 | AASSRAT |
| ZYOEPO-C07; CLDR3 | 30 | QQYGSSPLYT |
| ZYOEPQ-E02, variable | 31 | QVQLQQWGAGLLKPSETLSLTCAVYGGSFSGYYWN |
| heavy chain | | WIRQPPGKGLEWIGEINHSGSTNYNPSLKSRVTIL |
| | | VDTSKNQFSLKLSSVTAADTAVYYCARNLYNWNLD |
| | | SWGQGTLVTVSS |
| ZYOEPQ-E02, variable | 32 | DIQMTQSPSSLSASVGDRVTITCRASQGIRNDLGW |
| light chain | | YQQKPGRAPKRLIYVASSLQSGVPSRESGSGSGTE |
| | | FTLTISSLQPEDFATYYCLQHNSYPRTFGQGTKVE |
| | | IK |
| ZYOEQD-E02, variable | 33 | QVQLQQWGAGLLKPSETLSLTCAVYGGSFSGYYWN |
| heavy chain, e.g., pre- | | WIRQPPGKGLEWIGEINHSGSTSYNPSLKSRVTIS |
| germlining | | VDTSKNQFSLKLSSVTAADTAVYYCARVLYNWNVD |
| | | SWGQGTLVTVSS |
| ZYOEQD-E02, variable | 34 | DIQMTQSPSSLSASVGDRVTITCRASQDIRNDVGW |
| light chain | | YQQKPGKAPKRLIYAASRLQSGVPSRESGSGSGTE |
| | | FTLTISSLQPEDFATYYCLQHNSYPRTFGQGTKVE |
| | | IK |
| ZYOEOB-F05, variable | 35 | QVQLQESGPGLVKPSQTLSLTCTVSDGSISSGGYY |
| heavy chain | | WSWIRQHPGKGLEWIGNIYYSGSTYYNPSLKSRVT |
| | | ISVDTSKNQFSLKLNSVTAADTAVYYCATEKALAT |
| | | VTPSGYENYYTVDVWGQGTTVTVSS |
| ZYOEOB-F05, variable | 36 | DIQLTQSPSFLSASVGDRVTITCWASQGISSYLAW |
| light chain | | YQQKPGKAPKLLIYAASTLQSGVPSRFSGSGSGTE |
| | | FTLTISSLQPEDFATYYCQHLNSYPLTFGGGTKVE |
| | | IK |
| ZYOE05-E07, variable | 37 | QVQLQESGPGLVKPSQTLSLTCTVSGGSISSGGYY |
| heavy chain | | WSWIRQHPGKGLEWIGNIYYSGSTYYNPSLKSRVT |
| | | ISVDTSKNQFSLKLSSVTAADTAVYYCAREKALAS |
| | | VIPSGYENYYVVDVWGQGTTVTVSS |
| ZYOE05-E07, variable | 38 | DIQLTQSPSFLSASVGGRVTITCWASQGIAGYLAW |
| light chain | | YQQKPGKAPKLLIYAASTLQSGVPSRFSGSGSGTE |
| | | FTLTISSLQPEDFATYYCQHLNSYPLTFGGGTKVE |
| | | IK |
| ZYOEPO-C07, variable | 39 | QVQLVESGGVLVKPGGSLRLSCAASGFTLSDYYMS |
| heavy chain | | WIRQAPGMGLEWVSYISSSGSTIYYTDSVKGRFTI |
| | | SRDSAKNSLYLQMNSLRAEDTAVYYCARDGVGEDY |
| | | WGQGTLVTVSS |
| ZYOEPO-C07, variable | 40 | EIVLTQSPGTLSLEPGERATLSCRASQSVSSSYLA |
| light chain | | WYQQKPGQSPRLLIYAASSRATGIPDRFSGSGSGT |
| | | DETLTISRLEPEDFAVYYCQQYGSSPLYTFGQGTK |
| | | LEIK |
| Maia heavy chain | 41 | ASTKGPSVFPLAPSSKSTSGGTAALGCLVKDYFPE |
| constant region, | | PVTVSWNSGALTSGVHTFPAVLQSSGLYSLSSVVT |
| cysteine insertion | | VPSSSLGTQTYICNVNHKPSNTKVDKRVEPKSCDK |
| underlined | | THTCPPCPAPELLGGPSCVFLFPPKPKDTLMISRT |
| | | PEVTCVVVDVSHEDPEVKENWYVDGVEVHNAKTKP |
| | | REEQYNSTYRVVSVLTVLHQDWLNGKEYKCKVSNK |
| | | ALPAPIEKTISKAKGQPREPQVYTLPPSREEMTKN |
| | | QVSLTCLVKGFYPSDIAVEWESNGQPENNYKTTPP |
| | | VLDSDGSFFLYSKLTVDKSRWQQGNVFSCSVMHEA |
| | | LHNHYTQKSLSLSPGK |
| light chain constant | 42 | RTVAAPSVFIFPPSDEQLKSGTASVVCLLNNFYPR |
| region | | EAKVQWKVDNALQSGNSQESVTEQDSKDSTYSLSS |
| | | TLTLSKADYEKHKVYACEVTHQGLSSPVTKSENRG |
| | | EC |
| ZYOEQD-E02, variable | 43 | QVQLQQWGAGLLKPSETLSLTCTVYGGSFSGYYWN |
| heavy chain, e.g., pre- | | WIRQPPGRGLEWIGEINHSGSTSYNPSLKSRITIS |
| germlining, e.g., | | IDTSKNQFSLKLSSVTAADTAVYYCARVLYNWNVD |
| variant of SEQ ID NO: | | SWGQGTLVTVSS |
| 33/ SEQ ID NO: 45 | | |
| ZYOEQD-E02, light chain | 44 | DIQMTQSPSSLSASVGDRVTITCRASQDIRNDVGW |
| | | YQQKPGKAPKRLIYAASRLQSGVPSRESGSGSGTE |
| | | FTLTISSLQPEDFATYYCLQHNSYPRTFGQGTKVE |
| | | IKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSEN |
| | | RGEC |
| EQD-E02 GL, variable | 45 | QVQLQQWGAGLLKPSETLSLACTVYGGSFSGYYWN |
| heavy chain, GL = | | WIRQPPGKGLEWIGEINHSGSTSYNPSLKSRVTIS |
| germlined | | VDTSKNQFSLKLSSVTAADTAVYYCARVLYNWNVD |
| | | SWGQGTLVTVSS |
| EQD-E02-GLY, variable | 46 | QVQLQQWGAGLLKPSETLSLACTVYGGSFSGYYWN |
| heavy chain, GLY = | | WIRQPPGKGLEWIGEIYHSGSTSYNPSLKSRVTIS |
| germlined with a Y | | VDTSKNQFSLKLSSVTAADTAVYYCARVLYNWNVD |
| substitution | | SWGQGTLVTVSS |
| EQD-E02-GLQ, variable | 47 | QVQLQQWGAGLLKPSETLSLACTVYGGSFSGYYWN |
| heavy chain, GLQ = | | WIRQPPGKGLEWIGEIQHSGSTSYNPSLKSRVTIS |
| germlined with a Q | | VDTSKNQFSLKLSSVTAADTAVYYCARVLYNWNVD |
| substitution | | SWGQGTLVTVSS |
| E02-GL-Maia-heavy | 48 | QVQLQQWGAGLLKPSETLSLACTVYGGSFSGYYWN |
| chain, cysteine | | WIRQPPGKGLEWIGEINHSGSTSYNPSLKSRVTIS |
| insertion underlined | | VDTSKNQFSLKLSSVTAADTAVYYCARVLYNWNVD |
| | | SWGQGTLVTVSSASTKGPSVFPLAPSSKSTSGGTA |
| | | ALGCLVKDYFPEPVTVSWNSGALTSGVHTFPAVLQ |
| | | SSGLYSLSSVVTVPSSSLGTQTYICNVNHKPSNTK |
| | | VDKRVEPKSCDKTHTCPPCPAPELLGGPSCVELFP |
| | | PKPKDTLMISRTPEVTCVVVDVSHEDPEVKENWYV |
| | | DGVEVHNAKTKPREEQYNSTYRVVSVLTVLHQDWL |
| | | NGKEYKCKVSNKALPAPIEKTISKAKGQPREPQVY |
| | | TLPPSREEMTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGK |
| E02-GLY-Maia-heavy | 49 | QVQLQQWGAGLLKPSETLSLACTVYGGSFSGYYWN |
| chain, GLY = germlined | | WIRQPPGKGLEWIGEIYHSGSTSYNPSLKSRVTIS |
| with a Y substitution | | VDTSKNQFSLKLSSVTAADTAVYYCARVLYNWNVD |
| | | SWGQGTLVTVSSASTKGPSVFPLAPSSKSTSGGTA |
| | | ALGCLVKDYFPEPVTVSWNSGALTSGVHTFPAVLQ |
| | | SSGLYSLSSVVTVPSSSLGTQTYICNVNHKPSNTK |
| | | VDKRVEPKSCDKTHTCPPCPAPELLGGPSCVELFP |
| | | PKPKDTLMISRTPEVTCVVVDVSHEDPEVKENWYV |
| | | DGVEVHNAKTKPREEQYNSTYRVVSVLTVLHQDWL |
| | | NGKEYKCKVSNKALPAPIEKTISKAKGQPREPQVY |
| | | TLPPSREEMTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGK |
| E02-GLQ-Maia-heavy | 50 | QVQLQQWGAGLLKPSETLSLACTVYGGSFSGYYWN |
| chain, GLQ = germlined | | WIRQPPGKGLEWIGEIQHSGSTSYNPSLKSRVTIS |
| with a Q substitution | | VDTSKNQFSLKLSSVTAADTAVYYCARVLYNWNVD |
| | | SWGQGTLVTVSSASTKGPSVFPLAPSSKSTSGGTA |
| | | ALGCLVKDYFPEPVTVSWNSGALTSGVHTFPAVLQ |
| | | SSGLYSLSSVVTVPSSSLGTQTYICNVNHKPSNTK |
| | | VDKRVEPKSCDKTHTCPPCPAPELLGGPSCVELEP |
| | | PKPKDTLMISRTPEVTCVVVDVSHEDPEVKENWYV |
| | | DGVEVHNAKTKPREEQYNSTYRVVSVLTVLHQDWL |
| | | NGKEYKCKVSNKALPAPIEKTISKAKGQPREPQVY |
| | | TLPPSREEMTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGK |
| E02-GL-WT-heavy chain | 51 | QVQLQQWGAGLLKPSETLSLACTVYGGSFSGYYWN |
| | | WIRQPPGKGLEWIGEINHSGSTSYNPSLKSRVTIS |
| | | VDTSKNQFSLKLSSVTAADTAVYYCARVLYNWNVD |
| | | SWGQGTLVTVSSASTKGPSVFPLAPSSKSTSGGTA |
| | | ALGCLVKDYFPEPVTVSWNSGALTSGVHTFPAVLQ |
| | | SSGLYSLSSVVTVPSSSLGTQTYICNVNHKPSNTK |
| | | VDKRVEPKSCDKTHTCPPCPAPELLGGPSVELEPP |
| | | KPKDTLMISRTPEVTCVVVDVSHEDPEVKENWYVD |
| | | GVEVHNAKTKPREEQYNSTYRVVSVLTVLHQDWLN |
| | | GKEYKCKVSNKALPAPIEKTISKAKGQPREPQVYT |
| | | LPPSREEMTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| heavy chain constant | 52 | ASTKGPSVFPLAPSSKSTSGGTAALGCLVKDYFPE |
| region | | PVTVSWNSGALTSGVHTFPAVLQSSGLYSLSSVVT |
| | | VPSSSLGTQTYICNVNHKPSNTKVDKRVEPKSCDK |
| | | THTCPPCPAPELLGGPSCVFLFPPKPKDTLMISRT |
| | | PEVTCVVVDVSHEDPEVKENWYVDGVEVHNAKTKP |
| | | REEQYNSTYRVVSVLTVLHQDWLNGKEYKCKVSNK |
| | | ALPAPIEKTISKAKGQPREPQVYTLPPSREEMTKN |
| | | QVSLTCLVKGFYPSDIAVEWESNGQPENNYKTTPP |
| | | VLDSDGSFFLYSKLTVDKSRWQQGNVFSCSVMHEA |
| | | LHNHYTQKSLSLSPGK |
| Human B7H4 nucleic acid | 53 | GCCACCatggcttccctggggcagatcctcttctg |
| sequence, including 5′ | | gagcataattagcatcatcattattctggctggag |
| and 3′ UTR | | caattgcactcatcattggctttggtatttcaggg |
| | | agacactccatcacagtcactactgtcgcctcagc |
| | | tgggaacattggggaggatggaatcctgagctgca |
| | | cttttgaacctgacatcaaactttctgatatcgtg |
| | | atacaatggctgaaggaaggtgttttaggcttggt |
| | | ccatgagttcaaagaaggcaaagatgagctgtcgg |
| | | agcaggatgaaatgttcagaggccggacagcagtg |
| | | tttgctgatcaagtgatagttggcaatgcctcttt |
| | | gcggctgaaaaacgtgcaactcacagatgctggca |
| | | cctacaaatgttatatcatcacttctaaaggcaag |
| | | gggaatgctaaccttgagtataaaactggagcctt |
| | | cagcatgccggaagtgaatgtggactataatgcca |
| | | gctcagagaccttgcggtgtgaggctccccgatgg |
| | | ttcccccagcccacagtggtctgggcatcccaagt |
| | | tgaccagggagccaacttctcggaagtctccaata |
| | | ccagctttgagctgaactctgagaatgtgaccatg |
| | | aaggttgtgtctgtgctctacaatgttacgatcaa |
| | | caacacatactcctgtatgattgaaaatgacattg |
| | | ccaaagcaacaggggatatcaaagtgacagaatcg |
| | | gagatcaaaaggcggagtcacctacagctgctaaa |
| | | ctcaaaggcttctctgtgtgtctcttctttctttg |
| | | ccatcagctgggcacttctgcctctcagcccttac |
| | | ctgatgctaaaaTAATAA |
| Human B7H4 nucleic acid | 54 | atggcttccctggggcagatcctcttctggagcat |
| sequence, coding | | aattagcatcatcattattctggctggagcaattg |
| sequence | | cactcatcattggctttggtatttcagggagacac |
| | | tccatcacagtcactactgtcgcctcagctgggaa |
| | | cattggggaggatggaatcctgagctgcacttttg |
| | | aacctgacatcaaactttctgatatcgtgatacaa |
| | | tggctgaaggaaggtgttttaggcttggtccatga |
| | | gttcaaagaaggcaaagatgagctgtcggagcagg |
| | | atgaaatgttcagaggccggacagcagtgtttgct |
| | | gatcaagtgatagttggcaatgcctctttgcggct |
| | | gaaaaacgtgcaactcacagatgctggcacctaca |
| | | aatgttatatcatcacttctaaaggcaaggggaat |
| | | gctaaccttgagtataaaactggagccttcagcat |
| | | gccggaagtgaatgtggactataatgccagctcag |
| | | agaccttgcggtgtgaggctccccgatggttcccc |
| | | cagcccacagtggtctgggcatcccaagttgacca |
| | | gggagccaacttctcggaagtctccaataccagct |
| | | ttgagctgaactctgagaatgtgaccatgaaggtt |
| | | gtgtctgtgctctacaatgttacgatcaacaacac |
| | | atactcctgtatgattgaaaatgacattgccaaag |
| | | caacaggggatatcaaagtgacagaatcggagatc |
| | | aaaaggcggagtcacctacagctgctaaactcaaa |
| | | ggcttctctgtgtgtctcttctttctttgccatca |
| | | gctgggcacttctgcctctcagcccttacctgatg |
| | | ctaaaa |
| Human B7H4 polypeptide | 55 | MASLGQILFWSIISIIIILAGAIALIIGFGISGRH |
| sequence; UniProt | | SITVTTVASAGNIGEDGILSCTFEPDIKLSDIVIQ |
| Accession No.: Q7Z7D3 | | WLKEGVLGLVHEFKEGKDELSEQDEMERGRTAVFA |
| | | DQVIVGNASLRLKNVQLTDAGTYKCYIITSKGKGN |
| | | ANLEYKTGAFSMPEVNVDYNASSETLRCEAPRWEP |
| | | QPTVVWASQVDQGANFSEVSNTSFELNSENVTMKV |
| | | VSVLYNVTINNTYSCMIENDIAKATGDIKVTESEI |
| | | KRRSHLQLLNSKASLCVSSFFAISWALLPLSPYLM |
| | | LK |

All references cited herein, including patents, patent applications, papers, textbooks and the like, and the references cited therein, to the extent that they are not already, are hereby incorporated herein by reference in their entirety.

Without limiting the disclosure, a number of aspects of the disclosure are described herein for purpose of illustration.

### EXAMPLES

The Examples that follow are illustrative of specific aspects of the disclosure, and various uses thereof. They are set forth for explanatory purposes only and should not be construed as limiting the scope of the disclosure in any way.

AZD8205 is provided for treatment in the Examples. AZD8205 is an ADC that targets B7-H4 (VTCN1), consisting of a human anti-B7-H4 antibody (“INT016”) that is conjugated, via a cleavable maleimide-PEG8-valine-alanine linker, to a TOP1i warhead (“AZ14170132”). AZD8205 has a DAR of about 8. The antibody component of AZD8205 binds to B7-H4 on the cell surface and promotes internalization of the ADC as well as its trafficking to the lysosome. Enzymatic cleavage of the linker releases the TOP1i warhead, which traps TOP1 onto the DNA and leads to the formation of double-strand DNA breaks. If unrepaired, the double-strand breaks ultimately lead to programmed cell death.

Preclinical pharmacological studies suggest that the primary mechanism of action of AZD8205 is intracellular delivery of the TOP1i warhead into B7-H4 positive tumor cells. In vitro assays demonstrated that AZD8205 specifically binds, and is cytotoxic to, B7-H4 expressing tumor cells but not B7-H4 negative cells. Results from an in vivo pharmacodynamic study using a human tumor xenograft mouse model showed a dose-dependent accumulation of AZD8205 and increased positive staining for γH2AX foci (an indicator of DNA double strand breaks), elevated cleaved caspase-3 and an overall decrease in epithelial cell density over time, suggesting that AZD8205 binds to B7-H4 on tumor cells, causing DNA damage and apoptotic cell death, consistent with the mechanism of action of its TOP1i warhead.

Subsequent in vivo efficacy studies of AZD8205 in human tumor cell line-derived xenograft and patient-derived xenograft models of triple-negative breast cancer further demonstrated that a single IV administration of AZD8205 resulted in antitumor efficacy that is both target- and dose-dependent. In a study evaluating patient-derived xenograft models of triple-negative breast cancer (N=26), a single IV dose of 1.25 mg/kg or 3.5 mg/kg resulted in effective tumor regression (reduction in tumor volume of 30% or more from baseline) in 12 of 26 (46%) and 18 of 26 (69%) of the models, respectively. To elucidate the relationship between B7-H4 expression and AZD8205 response, immunohistochemistry and image analysis techniques were used to quantify B7-H4 expression in each patient-derived xenograft model. A statistically significant association between B7-H4 expression and antitumor efficacy was observed following treatment with 1.25 mg/kg AZD8205, suggesting that elevated levels of B7-H4 are associated with AZD8205 response.

### Example 1: Starting Dose Justification

As described in the Examples below, AZD8205 monotherapy is being investigated at dose levels ranging from 0.8 mg/kg to 3.2 mg/kg Q3W intravenously. The recommended starting dose for single agent AZD8205 of 0.8 mg/kg is based on the results of a good laboratory practice (GLP) toxicology study in cynomolgus monkeys, scaling of pharmacokinetics (PK) to humans from cynomolgus monkeys, and FDA regulatory guidelines. Dose increments and clinically efficacious dose range (0.8 to 2.4 mg/kg) were determined, and modeled exposure were estimated. The highest nonseverely toxic dose (HNSTD) of AZD8205 was identified as 15 mg/kg every 3 weeks (Q3W) for a total of 2 doses in a GLP study in cynomolgus monkeys, and the human equivalent dose (HED) for this HNSTD was calculated to be 4.8 mg/kg based on “US FDA Guidance for Industry: estimating the maximum safe starting dose in initial clinical trials for therapeutics in adult healthy volunteers.” The selection of the clinical starting dose of 0.8 mg/kg Q3W for first-time-in-human (FTIH) study is based on one-sixth the HED of 4.8 mg/kg. At a dose of 0.8 mg/kg AZD8205 Q3W, administered by 1 hour intravenous infusion, the steady state plasma exposures of AZD8205 are predicted to be a Cₘₐₓ of 16 g/mL and an AUC_{0-21 d} of 77 μg·d/mL.

This approach for identification of the starting dose for ADCs has generally been shown to result in an acceptable balance of safety and efficient dose escalation in a FTIH trial. The human starting dose of 0.8 mg/kg is expected to have acceptable HNSTD-based safety margins: 6.05 based on HED, 20.4 based on the Cₘₐₓ and 11.2 based on AUC.

### Example 2: Study Design and Definitions

This first-time-in-human (FTIH) study is conducted within the context of a master protocol to evaluate the safety, tolerability, and preliminary efficacy of AZD8205 initially as monotherapy and after defining the recommended phase 2 dose (RP2D), in combinations with other anticancer agents in subjects with B7-H4 expressing advanced or metastatic solid malignancies. The protocol also explores potential biological activity of AZD8205 by characterizing pharmacokinetics (PK), pharmacodynamics (PDx), exploratory biomarkers, and anti-tumor activity.

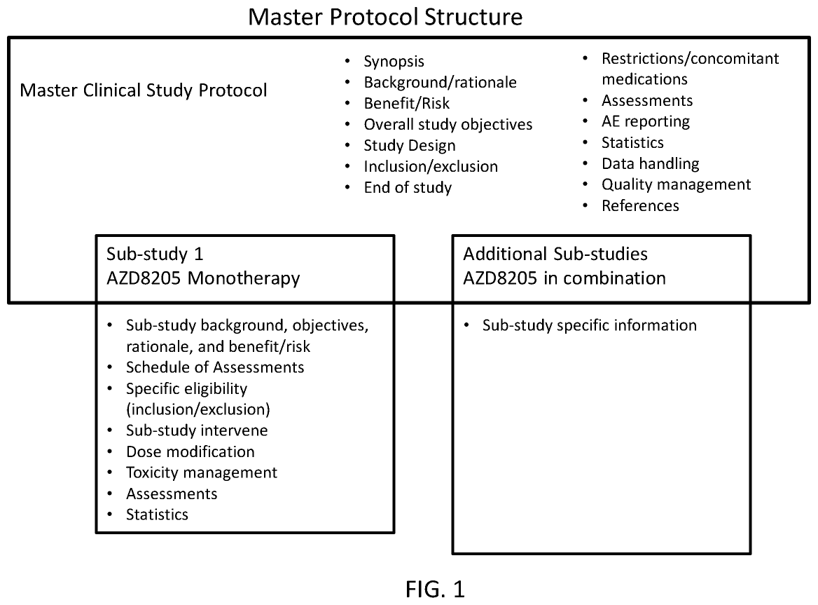

FIG. 1 shows the master protocol design. As shown in FIG. 1, information relating to the overall study, including study objectives, rationale, general inclusion and exclusion criteria, safety assessments, and adverse event (AE) reporting that are common for all sub-studies can be found in the master protocol. Sub-study-specific information, including planned dose levels and justifications, toxicity management, and dose modifications can be found in the relevant sub-study.

The master protocol consists of several sub-studies evaluating AZD8205. Sub-studies for combination treatments are added after defining monotherapy recommended Phase 2 dose (RP2D) based on emerging supportive preclinical and/or clinical data and scientific rationale. Each sub-study contains dose escalation (e.g., Part A) and dose expansion (e.g., Part B). The aim of the dose escalation (Part A) of each sub-study is to determine the safety, tolerability, RP2D and/or the maximum tolerated dose (MTD) of AZD8205 in subjects with B7-H4 expressing tumors. All subjects selected for this study had tumors where at least 25% of the cells in their tumor biopsy were positive for B7-H4, as described in Example 9. The RP2D is selected by pooling and evaluating all available data, including appropriate PK, PD, efficacy, safety, and tolerability. The aim of the dose expansion (Part B) of each sub-study is to evaluate anti-tumor activity of AZD8205. Initiation of Part B for each sub-study depends on the evaluation of safety outcomes in Part A, and determination of a RP2D for AZD8205 monotherapy and for each combination.

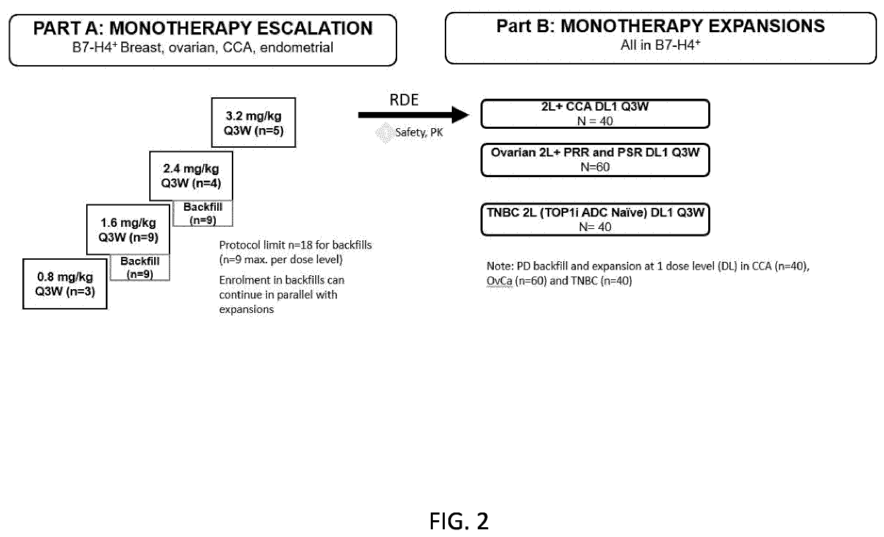

FIG. 2 shows a study flow of Sub-study 1 outlined in the Examples below. Key aspects of the study, such as the pre-screening selection process and starting dose of AZD8205 in Sub-study 1, the dose escalation, stopping criteria, and cohort sizes for Part A of all sub-studies, are based upon accepted methodology for Phase I/II oncology studies. Furthermore, blood samples are collected to enable the characterization of AZD8205 PK and, if appropriate, the investigation of additional metabolites.

As part of the clinical drug development program for AZD8205, PDx and exploratory biomarker (DNA, RNA, proteins or metabolites) profiles are investigated, and their relationship to drug effect is studied. The potential benefits of this exploratory research include (1) identification of subjects most likely to benefit from treatment; (2) prediction of which subjects may not respond to treatment; (3) identification of adverse reactions related to drug exposure; and (4) characterization of emerging toxicities.

In summary, the examples provide the information and understanding required to deliver safe and effective new medicines to subjects who have tumors that express B7-H4. Definitions—The following terms are used in the study described in the Examples below.

Dose-limiting Toxicity (DLT). The DLTs are evaluated during Part A (dose escalation). The DLT-evaluation period is 21 days from the first dose of AZD8205 on Cycle 1 Day 1 or up to and including the planned end of Cycle 1 if an alternative dosing schedule is explored. A DLT is defined as any ≥Grade 3 treatment-emergent AE that occurs during the DLT evaluation period, not attributable to primary disease or disease-related processes or intercurrent illnesses, with modifications or exceptions.

Maximum Tolerated Dose (MTD). The MTD is defined as the highest dose with the target DLT probability of 30%. The MTD is determined by isotonic regression analysis applied to DLT rates observed during the dose-escalation phase, using the modified toxicity probability interval-2 (mTPI-2) method (Guo et al 2017).

Efficacy Assessments. Tumor response is assessed by RECIST v1.1 (Eisenhauer et al, 2009) according to the schedule.

Tumor Evaluation. Tumor assessments use images from computerized tomography (CT, preferred) or magnetic resonance imaging (MRI), with IV contrast, of the chest, abdomen, and pelvis, and additionally investigate areas that may be involved based on signs and symptoms of individual subjects, collected during screening/baseline and at regular (follow-up) intervals during study intervention. Brain scans (MRI preferred) are mandatory at screening/baseline for all subjects in this study. Post baseline MRI of the brain only has to be performed in subjects with brain metastases at baseline, while subjects without brain metastases do not need additional brain scans for subsequent tumor assessments unless clinically indicated. The imaging modality used for baseline tumor assessment is kept the same consistently at each subsequent follow-up assessment throughout the study if possible.

Screening/baseline imaging is performed no more than 28 days before start of study intervention and ideally is performed as close as possible to and prior to the start of study intervention. Scans obtained as part of standard clinical practice, prior to informed consent, but within the 28-day period are acceptable. Tumor assessments are performed per the schedule in the respective sub-studies until objective disease progression as defined by RECIST v1.1 and assessed by the Investigator, or withdrawal of consent. Response (complete response (CR) or partial response (PR)) is confirmed by a repeat, consecutive scan at least 4 weeks after the first documentation of response.

Tumor markers are not used for tumor response assessments as per RECIST v1.1. Tumor markers are collected for separate analyses. However, the results will not contribute to tumor response based on RECIST v1.1 assessment.

Eastern Cooperative Oncology Group (ECOG) Performance Status (PS). The subject's performance status is assessed as detailed in the schedule of the respective sub-study using the ECOG PS scale.

Adverse Events. An adverse event (AE) is the development of any untoward medical occurrence in a subject or clinical study subject administered a medicinal product and which does not necessarily have a causal relationship with this treatment. An AE can therefore be any unfavorable and unintended sign (e.g., an abnormal laboratory finding), symptom (for example nausea, chest pain), or disease temporally associated with the use of a medicinal product, whether or not considered related to the medicinal product.

The term AE is used to include both serious and non-serious AEs and can include a deterioration of a pre-existing medical occurrence. An AE may occur at any time, including run-in or washout periods, even if no study intervention has been administered.

Serious Adverse Events. A serious adverse event (SAE) is an AE occurring during any study phase (i.e., run-in, treatment, washout, follow-up), that fulfils one or more of the following criteria: (1) results in death; (2) is immediately life-threatening; (3) requires subject hospitalization or prolongation of existing hospitalization; (4) results in persistent or significant disability or incapacity; (5) is a congenital anomaly or birth defect; (6) is an important medical event that may jeopardize the subject or may require medical treatment to prevent one of the outcomes listed above.

Disease Progression. Disease progression can be considered as a worsening of a subject's condition attributable to the disease for which the investigational product is being studied. It may be an increase in the severity of the disease under study and/or increases in the symptoms of the disease. The development of new or progression of existing metastasis to the primary cancer under study should be considered as disease progression and not an AE. Events that are unequivocally due to disease progression should not be reported as AEs during the study.

New Cancers. The development of a new cancer should be regarded as an SAE. New primary cancers are those that are not the primary reason for the administration of the study intervention and have been identified after the subject's inclusion in this study. They do not include metastases of the original cancer.

### Example 3: Formulation and Administration of AZD8205

This example shows the formulation and administration of AZD8205. AZD8205 is supplied in Part A and Part B as either a lyophilized or liquid product.

Lyophilized AZD8205 is supplied in 20R amber vials as a sterile lyophilized solid containing 100 mg AZD8205 (nominal) per vial. The AZD8205 investigational product (IP) must be stored at 2° C. to 8° C. (refrigerated) prior to use and protected from light exposure. AZD8205 vials must not be frozen.

Liquid AZD8205 is supplied as an intravenous bag protectant (IVBP) solution to ensure compatibility of AZD8205 to the IV infusion components and diluent solution. The IVBP is stored at 2° C. to 8° C. (refrigerated). The lyophilized AZD8205 must not be reconstituted with the IVBP solution. Sterile water for injection is used for reconstitution.

All IPs should be kept in a secure and dry place. Vials should be stored protected from light at 2° C. to 8° C. (refrigerated) and must not be frozen.

Weight-based doses are calculated using the following formula:

$$Dose ( mL ) = \frac{subject weight ( kg ) \times dose level ( \frac{mg}{kg} )}{Investigational Product concentration ( \frac{mg}{mL} )}$$

### Example 4: Initial Risk/Benefit Assessment

Studies are conducted to assess the risk of AZD8205 administration and provide appropriate AZD8205 dose for Part A and Part B.

Preclinical toxicology studies assessing the safety of AZD8205 showed that exposure to AZD8205 in cynomolgus monkeys at levels greater than the anticipated clinical efficacious range elicited changes consistent with that of other marketed TOP1i ADCs. Key target organs of toxicity were those with rapid cell turnovers (e.g., bone marrow and testes).

In the non-GLP dose range finding study, male cynomolgus monkeys were exposed to AZD8205 at dose levels of 15, 20, 22.5, and 25 mg/kg (IV Q3W×2). Findings were as anticipated and consistent with cytotoxic TOP1 inhibitors. Dose levels ≥20 mg/kg were not tolerated. At dose levels ≥20 mg/kg, in addition to signs of moribundity, there were moderate to marked effects on the hematopoietic system (most notably decreased lymphocytes, neutrophils and reticulocytes) with histological correlates of moderate to marked bone marrow hypocellularity and decreased cellularity of other lymphoid organs; gastrointestinal tract toxicity was noted in the large and small intestinal mucosa (minimal to marked degeneration and regeneration) and was considered a key contributor to the moribund condition of the animals.

Other organs showing AZD8205 treatment-related pharmacological effects included reversible mild to moderate histopathological findings in the kidney, liver, pancreatic islets, and testes. Some of these treatment-related findings were also indicative of stress/poor and deteriorating clinical condition.

Clinical chemistry changes were noted in the liver and kidneys, including mild, transient, increase in liver enzymes (2-fold over control ALT/AST levels on Day 4 to 6 only, but without a histopathological correlate) and marked increase in renal enzymes that correlated with clinical observation of dehydration. All changes were recoverable before scheduled necropsy on Day 43. In surviving animals (2/3 at 22.5 mg/kg and 3/3 at 15 mg/kg), there were also effects on the hematopoietic system that were only minimal to mild in severity. The only treatment-related microscopic findings were noted in the tests, again consistent with the mechanism of action (MOA) of the TOP1 inhibitor, of animals dosed at 22.5 mg/kg IV Q3W×2. At 15 mg/kg, there were no histopathological findings in any organ examined.

In the pivotal GLP 6-week repeat dose (Q3W×2) IV study with a 6-week recovery period in the cynomolgus monkey, dose levels up to and including 15 mg/kg did not reveal any adverse findings (the maximum dose tested and the HNSTD). All major organs and functions (respiratory, cardiovascular, renal, liver, and nervous system) were assessed and no AZD8205-related clinical signs or effects were noted on survival, local (skin) tolerance, body weights, electrocardiogram, blood pressure, respiration rate, ophthalmoscopy, neurological endpoints, clinical chemistry, urinalysis, systemic cytokine release or coagulation parameters. The only treatment-related changes were observed at dose levels ≥10 mg/kg and were associated with anticipated pharmacological activity of AZD8205 on the hematopoietic system and included minimal and transient effects on some red cell parameters that were reversible during the 6-week recovery phase.

The pivotal GLP AZD8205 toxicity study in male and female cynomolgus monkeys found no evidence of liver enzyme elevations, hypersensitivity/anaphylaxis or infusion-related reactions at dose levels up to and including 15 mg/kg (systemic exposures of mean combined sex AUC of 963 μg·d/mL and Cₘₐₓ of 323 g/mL). It is anticipated that these values will provide approximately 13 and 20 fold margins of safety over the minimum predicted clinical efficacious AUC (77 μg·d/mL) and Cₘₐₓ (16 g/mL) levels at the starting dose in this study, respectively. Where hepatic enzyme changes were noted in the dose range finding study, they were minimal (circa 2 fold), transient (noted on 1 or 2 days in some animals), not associated with hepatocellular necrosis and fully recoverable by the end of dosing in animals at 15 mg/kg AZD8205.

### Example 5: Study Subjects

This Example provides criteria for selecting subjects for the Part A and Part B study of Sub-study 1.

Inclusion criteria for Part A and Part B:
1. Must be 18 years of age or over when enrolled in the study.
2. Eastern Cooperative Oncology Group (ECOG) performance status of 0 or 1 at enrollment.
3. Subjects with relapsed/metastatic solid tumors must have received prior adequate SoC therapy for their tumor type and stage of disease or where a clinical trial is the best option for the next treatment based on response and/or tolerability to prior therapy.
4. Subjects must have measurable disease per RECIST v1.1, as defined by at least 1 lesion that can be accurately measured at baseline as ≥10 mm at the longest diameter (except lymph nodes which must have a short axis ≥15 mm) with CT or MRI, which is suitable for accurate repeated measurements. Previously irradiated lesions or a lesion in the field of radiation should not be used as target lesion unless the lesion(s) has/have demonstrated unequivocal disease progression by RECIST v1.1. Target lesions should not be used for the baseline tumor biopsy, unless there are no other lesions suitable for biopsy and they fulfill predetermined requirements.
5. Predicted life expectancy ≥12 weeks.
6. Adequate organ function as defined in Table 2.

TABLE 2
Criteria for Adequate Organ and Marrow Function

| Type | Parameter | Value |
|---|---|---|
| Hematological | Hemoglobin | ≥9.0 g/dL (5.59 mmol/L) with no blood |
| | | transfusion within 28 days prior to first |
| | | dose |
| | Absolute neutrophil count | ≥1.5 × 10⁹/L (1500 per mm³) |
| | Platelet count | ≥100 × 10⁹/L (100000 per mm³) |
| | Prothrombin time-INR and | ≤1.5 × institutional ULN |
| | aPTT | |
| Hepatic | Total bilirubin | ≤1.5 × institutional ULN in the absence |
| | | of Gilbert's Syndrome |
| | | ≤3 × institutional ULN if the subject |
| | | has Gilbert's syndrome |
| | Alanine aminotransferase and | ≤3 × institutional ULN, unless liver |
| | aspartate aminotransferase | metastases are present in which case |
| | | must be ≤5 × ULN |
| Renal | CrCl (calculated using the | Creatinine ≤1 · 5 × ULN or CrCl ≥60 |
| | Cockcroft and Gault equation | mL/min (by Cockcroft-Gault or 24-hour |
| | (Cockcroft and Gault 1976) | urine collection) |

aPTT = partial prothrombin time; CrCl = creatinine clearance; D1C1 = Day 1 Cycle 1; INR = international normalized ratio; ULN = upper limit of normal.

### Example 6: Part A Dose Escalation

This Example describes the Part A dose escalation study to determine AZD8205 dosing for Part B.

This sub-study is being conducted to explore the safety and tolerability of AZD8205 as a monotherapy and to evaluate the anti-tumor activity of AZD8205 in breast, ovarian, endometrial, and biliary tract cancers. AZD8205 is administered intravenously Q3W. The study is conducted in the following phases applicable to both parts A and B:
1. Pre-screening phase: all subjects provide a baseline tissue sample for B7-H4 analysis. Subjects with tumors that express B7-H4 enter the next phase.
2. Screening phase: subjects are assessed for study intervention eligibility. Only subjects who meet all of the inclusion criteria and none of the exclusion criteria are assigned to the study.
3. Treatment phase: subjects continue to receive study intervention until one of the discontinuation criteria are met, including any AE contraindicates further dosing or an AE, or radiological progression.
4. Follow-up phase: all subjects are followed for survival.

Part A (dose escalation) evaluates AZD8205 in approximately 60 subjects with positive levels of B7-H4 tumor expression to determine an MTD and/or RP2D. The RP2D is selected by pooling and evaluating all available data, including appropriate PK, PD, efficacy, safety, and tolerability. Based on safety, PK, and PDx data, the SRC may recommend intermediate dose levels and/or alternative dosing schedules and longer treatment intervals. Based on emerging data, any dose level not exceeding the MTD may be expanded by up to an additional 18 subjects (referred to as the PDx backfill cohorts, 36 subjects in total). Subjects enrolled into the PDx backfill cohorts do not impact dosing decisions made by the SRC based on the mTPI-2 algorithm. The PDx backfill cohorts may enroll specific tumor types with mandatory pre-treatment and on-treatment tumor biopsies. These PDx backfill cohorts provide additional PDx and safety data that could inform optimal dose-level selection for expansion based on the available data.

In Part A, four dose levels of 0.8 mg/kg, 1.6 mg/kg, 2.4 mg/kg and 3.2 mg/kg were administered Q3W via IV.

Following an initial screening period of up to 28 days, the Part A intervention period was scheduled. The Part A intervention period includes up to or over 20 cycles, with each cycle 21 days or 28 days, and AZD8205 is administered every three weeks (Q3W), i.e., once every cycle of 21 days or every cycle of 28 days.

Eligible subjects receive AZD8205 Q3W administered via intravenous (IV) infusion at the selected dose starting on Cycle 1 Day 1 for up to or over 20 cycles. Subjects are treated with study intervention until disease progression, unacceptable toxicity, investigator's decision, completion of the maximum of treatment cycles, or withdrawal of consent. All subjects are followed for survival until the end of the study.

After the invention period, the subjects are further evaluated for disease progression, end of treatment condition, and follow-up condition.

### Example 7: Part B Dose Expansion

Part B study phase in this Example was conducted using a 2.4 mg/kg dose of AZD8205 for studying dose expansion.

The objectives and endpoints are described above in Example 6.

Part B (dose expansion) is initiated to investigate preliminary efficacy and build on safety data in approximately 220 subjects from specific populations. Four expansion cohorts are planned pending review of emerging data from Part A:
Approximately 40 subjects in the BTC cohort (Cohort B1)
Approximately 60 subjects in the ovarian cancer cohort (Cohort B2)
   Approximately 30 subjects with PRR (Cohort B2A)
   Approximately 30 subjects with PSR (Cohort B2B)
Approximately 90 subjects across the HER2-negative (IHC: 0, 1+, 2+/ISH−, as defined by the most recent ASCO/CAP guidelines) breast cancer cohorts (Cohort B3):
   Approximately 30 subjects with HR+, HER2-negative breast cancer (Cohort B3A)
   Approximately 30 subjects with TNBC (Cohort B3B)
   Approximately 30 subjects with HR±, HER2-negative breast cancer who have been previously treated with TOP1i ADC (Cohort B3C)
Approximately 30 subjects in the endometrial carcinoma cohort (Cohort B4).

Each expansion cohort may be initiated at more than one dose level or schedule, depending on emerging data. Expansion cohorts may be opened concurrently or independently with a parallel or staggered approach. If a new dosing schedule is explored, dose modifications will be agreed and will be based on previously established safe exposure ranges. The overall sample size for Part B could increase up to 440 subjects, if it is decided to investigate two dose levels/schedules within an expansion cohort. In such cases, subjects may be randomized to the selected dose levels/schedules; allocation will not be blinded. Randomization will occur on Day 1.

Safety and tolerability data for all subjects will be evaluated regularly and subjects' clinical response status will be classified according to RECIST v1.1.

### Example 8: Preliminary Part A and Part B Results

35 subjects have been evaluated in the Part A and Part B portions of the study described in the Examples above, and evaluations of 16 subjects are ongoing.

Table 3 shows the demographics of the subjects evaluated in Part A and Part B.

TABLE 3
Demographics of Subjects Evaluated

| | | Overall ITT (N = 31) |
|---|---|---|
| Age median | | 56 (24-88) |
| Sex | Female | 28 |
| | Male | 3 |

Preliminary PK data are provided. Data are available from n=3 pts (0.8 mg/kg), n=9 pts (1.6 mg/kg), n=9 (2.4 mg/Kg) and n=5 pts (3.2 mg/Kg). According to the PK data, total AZD8205 ADC and total antibody concentrations are comparable at all dose levels tested, indicating that warhead conjugation is stable in subjects. Particularly, AZD8205 exposure is consistent with preclinical predictions, proportional increase with dose, with a half-life=4.9-7.2 days. Further, AZD8205 exposure at 1.6 and 2.4 mg/kg (AUC(0-21) 143.5±39.2 and 190.9±10.1 day*μg/mL, respectively) is within predicted efficacious range (80-230 day*μg/mL). Unconjugated warhead exposure in plasma is within 1.2-fold of predicted AUC.

Computer tomography data suggest that AZD8205 has activity in breast cancer (BC), ovarian cancer (OC), and endometrial cancer (EC). Particularly, 4 subjects were shown with both reduction of tumor markers+shrinkage (>30%) in target lesions (1uPR & 3cPR). 18 subjects were shown with SD as BoR by RECIST (including 4 ongoing subjects with tumor shrinkage ≥20%). Tumor markers decreased >50% in 10/24 evaluable pts across all dose levels in OC, BC and EC. ctDNA molecular measurement shows response in N=14/21 evaluable subjects (~67%)-N=5/8 subjects in Dose Level 2 (DL2) (63%), N=6/9 subjects in Dose Level 3 (DL3) (67%), 3/4 subjects in Dose Level 4 (DL4) (75%), with patterns largely consistent with clinical response and tumor markers.

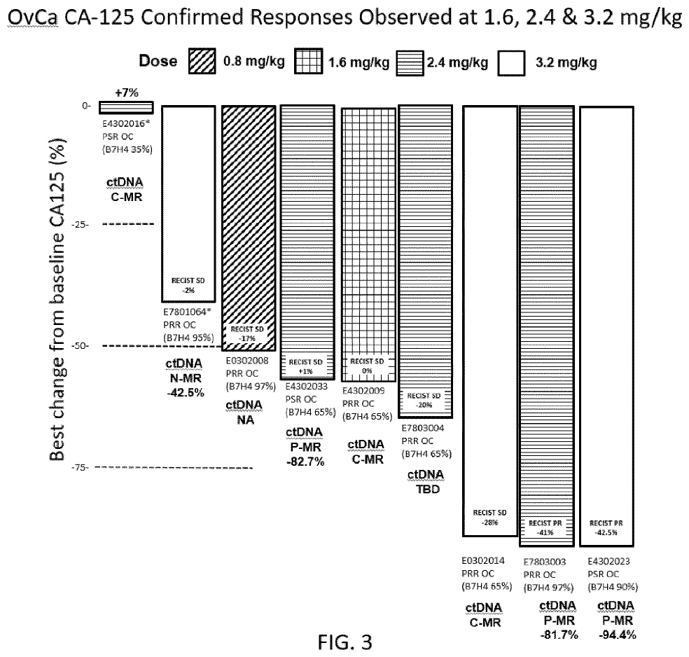

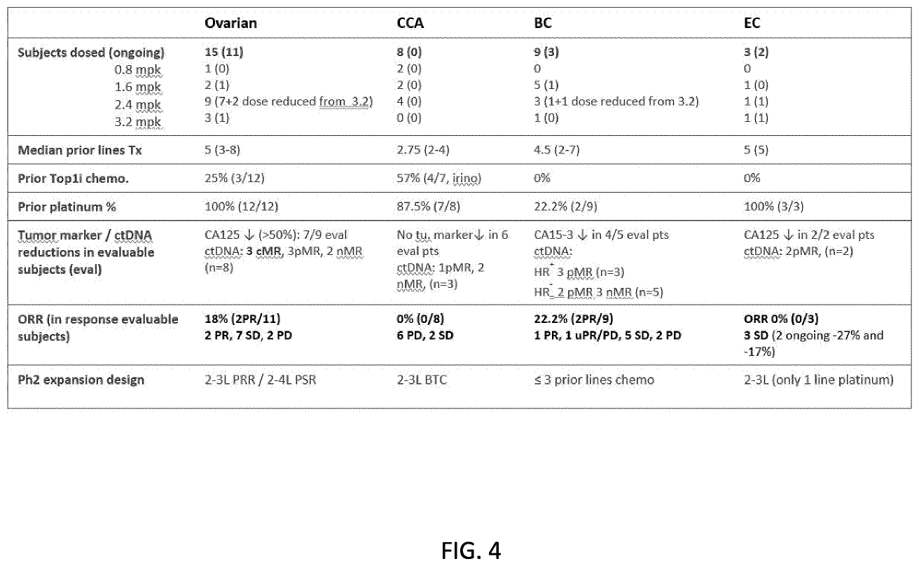

OvCa subjects with measurable CA125 (>2×ULN) are plotted in FIG. 3, where responses to AZD8205 at 1.6, 2.4, and 3.2 mg/kg are confirmed. ctDNA molecular response were observed in about 67% evaluable subjects (n=14/21) at 1.6, 2.4, and 3.2 mg/kg. The ctDNA changes largely mirror clinical response with greater ctDNA decrease observed in subjects deriving benefit. Emerging preclinical/clinical efficacy results are shown in FIG. 4.

### Example 9: Determination of B7-H4 Cutoff for Selected Subjects

During the early stages of the first in humans study described in the above Examples, it was observed that a higher overall response rate (ORR) was seen in subjects with tumors having higher levels of B7-H4 expression were more responsive to treatment with AZD8205. ORRs for all subjects versus B7-H4 expressing subjects are summarized in Table 7 below.

TABLE 7
ORRs for All Subjects Compared to B7-H4 Expressors

| | Cholangio- | | Triple-Negative |
|---|---|---|---|
| | carcinoma | Ovarian | Breast Cancer |
| | (CCA) | Cancer | (TNBC) |
| ORR - All Samples | 14% | 47% | 69% |
| ORR - B7-H4 | 21% | 64% | 79% |
| Expressors | | | |

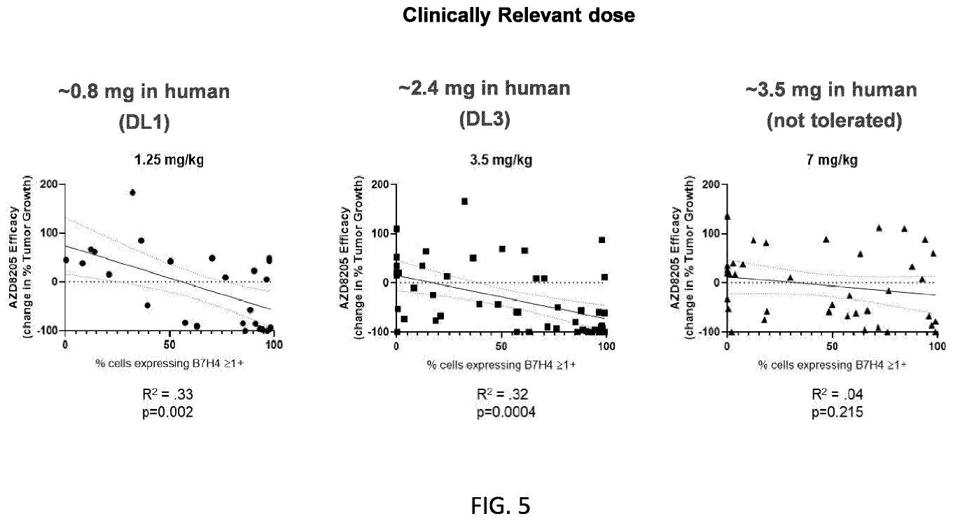

In order to determine a B7-H4 expression cutoff for continued human studies, mouse xenograft studies were performed for CCA, ovarian cancer and TNBC. Either athymic nude mice (Fox1^{nu}) or NOD-SCID mice were xenografted with tumors 21 days prior to administration of AZD8205. AZD8205 was administered at day 0 as a single intravenous dose. The change in tumor growth was monitored 28 days after treatment. Plots of the change in percent tumor growth versus the percentage of cells expressing B7-H4 are shown in FIG. 5.

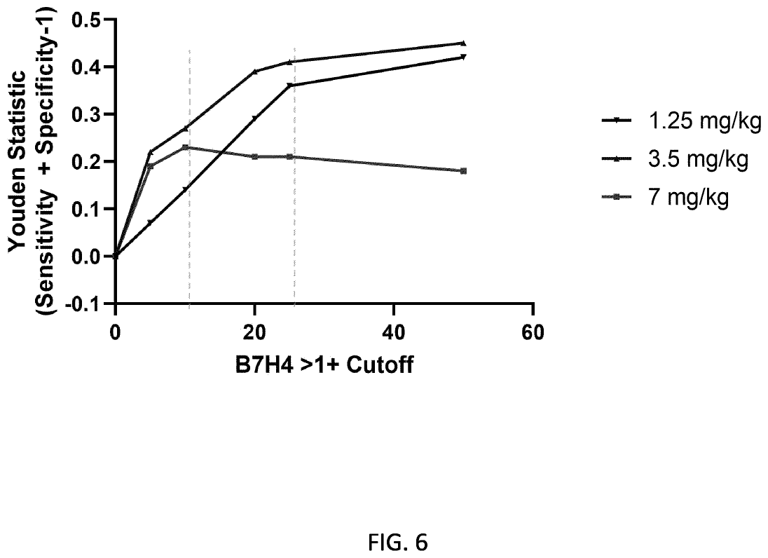

Receiver operating characteristic (ROC) curves were determined for each dose and the curves were used to calculate Youden statistics at each dose. The Youden statistic is plotted versus the percentage of B7-H4 cells as shown in FIG. 6. A Youden statistic of 1 represents a perfect biomarker while a statistic of 0 means the biomarker is non-differentiating. As can be seen in FIG. 6, the Youden statistic plateaued at a B7-H4 percentage of 25% or greater. Thus, a 25% B7-H4 expression cutoff for use to evaluate the doses being used.

### Example 10: Overall Response Rates in Heavily Pre-Treated Subjects

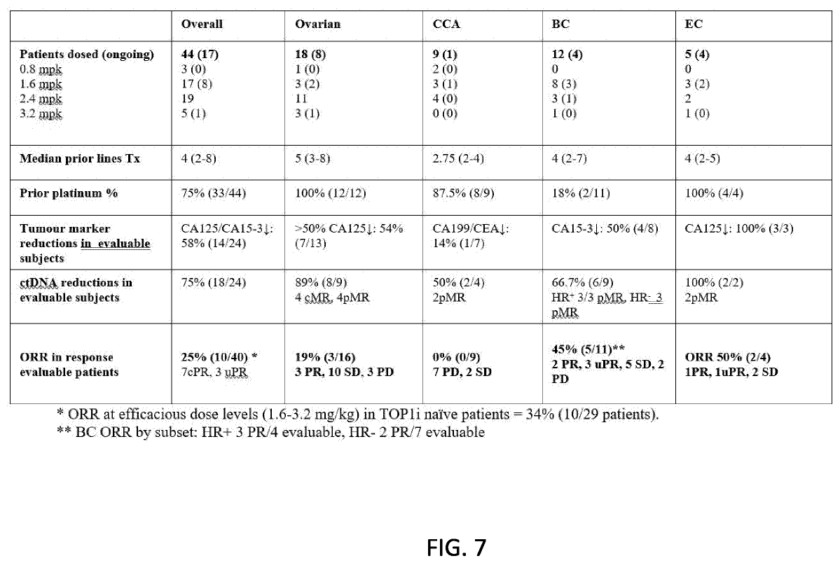

A total of 44 heavily pre-treated subjects from the study described in the Examples above were evaluated for objective response rate (ORR). As shown in FIG. 7 as part of a second data cut in view of FIG. 4, the 44 subjects include 18 subjects having ovarian cancer, 9 subjects having cholangiocarcinoma (CCA), 12 subjects having breast cancer (BC), and 5 subjects having endometrial cancer (EC). Of these 44 subjects, 40 were evaluable for ORR. Some of the subjects were previously treated with platinum chemotherapy as indicated in FIG. 7. As shown in the FIG. 7, for those subjects having significant tumor marker reduction and/or ctDNA reduction, the average ORR is about 25%.

1. A method of treating cancer in a subject, comprising administering to the subject an antibody-drug conjugate (ADC) that specifically binds to B7-H4 in an amount from about 0.8 mg/kg to about 4.8 mg/kg, the ADC comprising:
i. an antibody or antigen binding fragment thereof which binds to a B7-H4 polypeptide, comprising:
a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof;
ii. a cleavable linker; and
iii. a cytotoxic agent.

2. The method of claim 1, wherein the amount of ADC administered is about 0.8 mg/kg, about 1.6 mg/kg, about 2.4 mg/kg, about 3.2 mg/kg, about 3.6 mg/kg or about 4.8 mg/kg.

3. The method of claim 1, wherein the amount of ADC administered is about 1.6 mg/kg.

4. The method of claim 1, wherein the amount of ADC administered is about 2.4 mg/kg.

5. The method of claim 1, wherein the amount of ADC administered is about 3.2 mg/kg.

6. The method of any one of claims 1-5, wherein the cancer comprises a cancer cell which expresses B7-H4.

7. The method of any one of claims 1-6, wherein the ADC is administered to the subject once every three weeks.

8. The method of any one of claims 1-7, wherein the cancer is selected from ovarian cancer, biliary tract cancer, breast cancer, pancreatic cancer, prostate cancer, hematological cancer, endometrial cancer, cholangiocarcinoma, NSCLC (squamous and/or adenocarcinoma), gastrointestinal cancer such as gastric cancer and colorectal cancer, and lung cancer.

9. The method of any one of claims 1 to 8, wherein the cancer is a breast cancer selected from hormone receptor-positive (HR+) breast cancer, human epidermal growth factor receptor 2 positive (HER2+) breast cancer, and triple negative breast cancer (TNBC).

10. The method of any one of claims 1 to 9, wherein the cancer is homologous recombination deficient (HRD) cancer.

11. The method of claim 10, wherein the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L.

12. The method of claim 11, wherein the mutation in an HRD gene is selected from BRCA1, BRCA2, and ATM.

13. The method of any one of claims 1 to 12, wherein the antibody or antigen binding fragment thereof comprises:
i. a variable heavy (VH) chain and a variable light (VL) chain comprising the amino acid sequence of SEQ ID NO: 45 and SEQ ID NO: 34, respectively, or a functional variant thereof;
ii. a VH chain and a VL chain comprising the amino acid sequence of SEQ ID NO: 33 and SEQ ID NO: 34, respectively, or a functional variant thereof;
iii. a VH chain and a VL chain comprising the amino acid sequence of SEQ ID NO: 43 and SEQ ID NO: 34, respectively, or a functional variant thereof;
iv. a VH chain and a VL chain comprising the amino acid sequence of SEQ ID NO: 46 and SEQ ID NO: 34, respectively, or a functional variant thereof;
v. a VH chain and a VL chain comprising the amino acid sequence of SEQ ID NO: 47 and SEQ ID NO: 34, respectively, or a functional variant thereof;
vi. a VH chain and a VL chain comprising the amino acid sequence of SEQ ID NO: 31, and SEQ ID NO: 32, respectively, or a functional variant thereof;
vii. a VH chain and a VL chain comprising the amino acid sequence of SEQ ID NO: 35 and SEQ ID NO: 36, respectively, or a functional variant thereof;
viii. a VH chain and a VL chain comprising the amino acid sequence of SEQ ID NO: 37 and SEQ ID NO: 38, respectively, or a functional variant thereof; or
ix. a VH chain and a VL chain comprising the amino acid sequence of SEQ ID NO: 39 and SEQ ID NO: 40, respectively, or a functional variant thereof.

14. The method of any one of claims 1 to 12, wherein the antibody or antigen binding fragment thereof comprises: a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof.

15. The method of any one of claims 1 to 13, wherein the antibody or antigen binding fragment thereof comprises a VH chain and a VL chain comprising the amino acid sequence of SEQ ID NO: 45 and SEQ ID NO: 34, respectively, or a functional variant thereof.

16. The method of any one of claims 1 to 15, wherein the antibody or antigen binding fragment thereof binds an OVCAR4 cell line.

17. The method of any one of claims 1 to 16, wherein the antibody or antigen binding fragment thereof comprises a heavy chain constant region comprising the amino acid sequence of SEQ ID NO: 41.

18. The method of any one of claims 1 to 16, wherein the antibody or antigen binding fragment thereof comprises a heavy chain constant region comprising the amino acid sequence of SEQ ID NO: 52.

19. The method of any one of claims 1 to 16, wherein the antibody or antigen binding fragment thereof comprises a light chain constant region comprising the amino acid sequence of SEQ ID NO: 42.

20. The method of any one of claims 1 to 16, wherein the antibody or antigen binding fragment thereof comprises a heavy chain comprising the amino acid sequence of SEQ ID NO: 51; and a light chain comprising the amino acid sequence of SEQ ID NO: 44.

21. The method of any one of claims 1 to 16, wherein the antibody or antigen binding fragment thereof comprises a heavy chain comprising the amino acid sequence of SEQ ID NO: 48; and a light chain comprising the amino acid sequence of SEQ ID NO: 44.

22. The method of any one of claims 1 to 21, wherein the antibody or antigen binding fragment thereof is a monoclonal antibody.

23. The method of any one of claims 1 to 22, wherein the antibody or antigen binding fragment thereof is a humanized monoclonal antibody.

24. The method of any one of claims 1 to 23, wherein the cleavable linker is an mp-PEG8-val-ala linker.

25. The method of any one of claims 1 to 24, wherein the cytotoxic agent is a topoisomerase inhibitor.

26. The method of claim 25, wherein the topoisomerase inhibitor is a compound of Formula A*

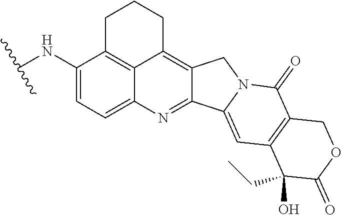 [SMILES: *Nc1ccc2nc3c(c4c2c1CCC4)Cn1c-3cc2c(c1=O)COC(=O)[C@]2(O)CC]

27. The method of any one of claims 1 to 26, wherein the ii) cleavable linker and iii) cytotoxic agent together are selected from the following compounds:

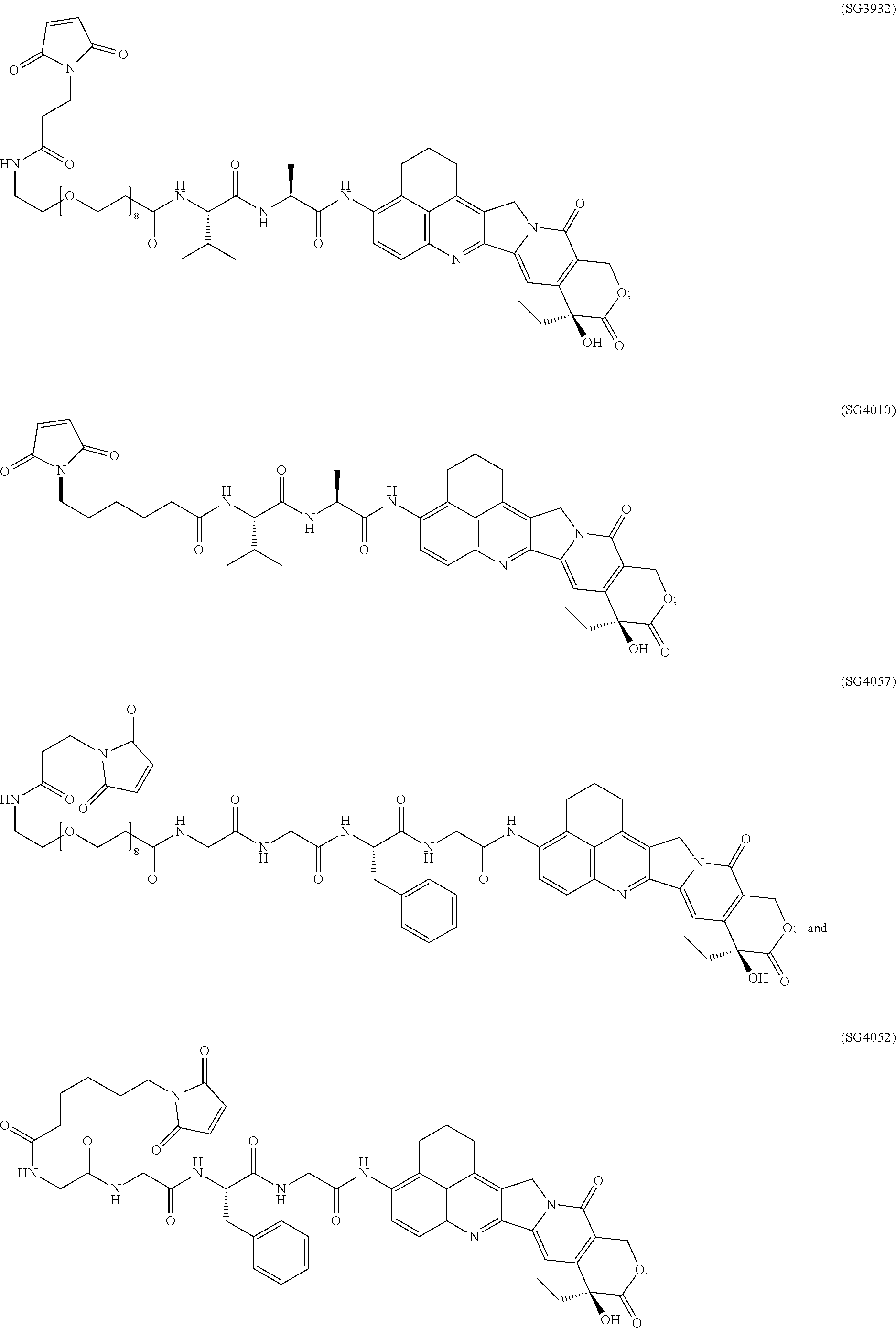

28. The method of claim 27, wherein the ii) linker and iii) cytotoxic agent together are the compound SG3932.

29. The method of any one of claims 1 to 28, wherein the ADC has a drug to antibody ratio (DAR) of between about 1 and about 8.

30. The method of any one of claims 1 to 28, wherein the ADC has a DAR of about 8.

31. A method of treating cancer in a subject, comprising administering to the subject an antibody-drug conjugate (ADC) that specifically binds to B7-H4 in an amount from about 0.8 mg/kg to about 4.8 mg/kg, the ADC comprising:
i. an antibody or antigen binding fragment thereof which binds to a B7-H4 polypeptide, comprising: a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof; and
ii. a cleavable linker and cytotoxic agent conjugated to the antibody or antigen binding fragment thereof having the formula:

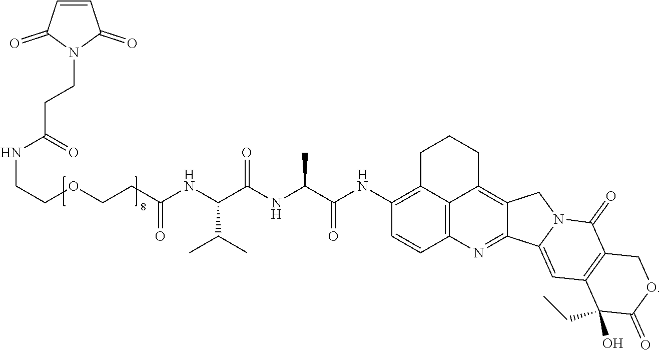

32. The method of claim 31, wherein the amount of ADC administered is about 0.8 mg/kg, about 1.6 mg/kg, about 2.4 mg/kg, about 3.2 mg/kg, about 3.6 mg/kg or about 4.8 mg/kg.

33. The method of claim 31, wherein the amount of ADC administered is about 1.6 mg/kg.

34. The method of claim 31, wherein the amount of ADC administered is about 2.4 mg/kg.

35. The method of claim 31, wherein the amount of ADC administered is about 3.2 mg/kg.

36. The method of any one of claims 31 to 35, wherein the antibody or antigen binding fragment thereof comprises a VH chain and a VL chain comprising the amino acid sequence of SEQ ID NO: 45 and SEQ ID NO: 34, respectively, or a functional variant thereof.

37. The method of any one of claims 31 to 36, wherein the cancer comprises a cancer cell which expresses B7-H4.

38. The method of any one of claims 31 to 37, wherein the ADC is administered to the subject once every three weeks.

39. The method of any one of claims 31 to 38, wherein the cancer is selected from ovarian cancer, biliary tract cancer, breast cancer, pancreatic cancer, prostate cancer, hematological cancer, endometrial cancer, cholangiocarcinoma, NSCLC (squamous and/or adenocarcinoma), gastrointestinal cancer such as gastric cancer and colorectal cancer, and lung cancer.

40. The method of any one of claims 31 to 39, wherein the cancer is a breast cancer selected from hormone receptor-positive (HR+) breast cancer, human epidermal growth factor receptor 2 positive (HER2+) breast cancer, and triple negative breast cancer (TNBC).

41. The method of any one of claims 31 to 40, wherein the cancer is homologous recombination deficient (HRD) cancer.

42. The method of claim 41, wherein the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L.

43. The method of claim 42, wherein the mutation in an HRD gene is selected from BRCA1, BRCA2, and ATM.

44. The method of any one of claims 1 to 43, wherein at least about 25% of cancer cells in the subject are B7-H4 positive cells, optionally wherein the cancer cells are assayed using immunohistochemistry (IHC).

45. A pharmaceutical composition comprising an antibody-drug conjugate (ADC) that specifically binds to B7-H4 which is administered in an amount from about 0.8 mg/kg to about 4.8 mg/kg, the ADC comprising:
i. an antibody or antigen binding fragment thereof which binds to a B7-H4 polypeptide, comprising: a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof; and
ii. a cleavable linker and cytotoxic agent conjugated to the antibody or antigen binding fragment thereof having the formula:

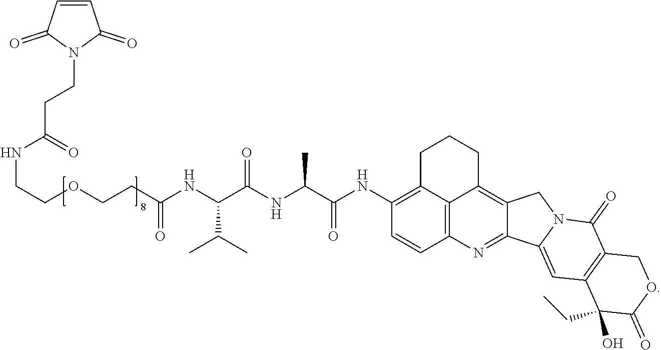

46. The pharmaceutical composition of claim 45, wherein the pharmaceutical composition is administered in an amount from about 0.8 mg/kg, about 1.6 mg/kg, about 2.4 mg/kg, about 3.2 mg/kg, about 3.6 mg/kg or about 4.8 mg/kg ADC.

47. The pharmaceutical composition of claim 45, wherein the pharmaceutical composition is administered in an amount of about 1.6 mg/kg ADC.

48. The pharmaceutical composition of claim 45, wherein the pharmaceutical composition is administered in an amount of about 2.4 mg/kg ADC.

49. The pharmaceutical composition of claim 45, wherein the pharmaceutical composition is administered in an amount of about 3.2 mg/kg ADC.

50. The pharmaceutical composition of any one of claims 45 to 49, wherein the antibody or antigen binding fragment of the ADC comprises a VH chain and a VL chain comprising the amino acid sequence of SEQ ID NO: 45 and SEQ ID NO: 34, respectively, or a functional variant thereof.

51. A pharmaceutical composition comprising an antibody-drug conjugate (ADC) that specifically binds to B7-H4 which is administered in an amount from about 25 mg to about 900 mg, the ADC comprising:
i. an antibody or antigen binding fragment thereof which binds to a B7-H4 polypeptide, comprising:
f) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
g) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
h) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
i) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
j) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof;
ii. a cleavable linker; and
iii. a cytotoxic agent.

52. The pharmaceutical composition of claim 51, wherein the amount of ADC administered is about 25 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, about 300 mg, about 350 mg, about 400 mg, about 450 mg, about 500 mg, about 600 mg, about 750 mg, or about 900 mg.

53. The pharmaceutical composition of claim 51 or 52, wherein the cleavable linker and cytotoxic agent combined have the formula:

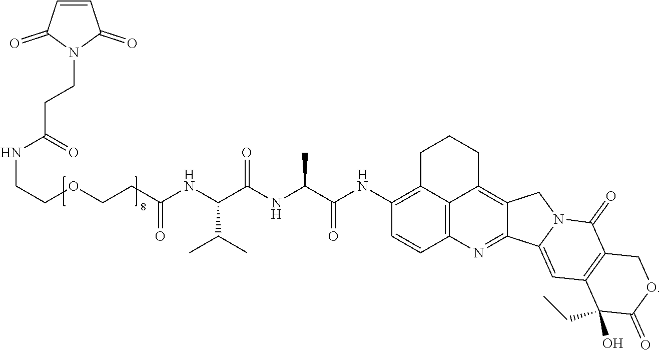

54. The pharmaceutical composition of any one of claims 51 to 53, wherein the antibody or antigen binding fragment of the ADC comprises a VH chain and a VL chain comprising the amino acid sequence of SEQ ID NO: 45 and SEQ ID NO: 34, respectively, or a functional variant thereof.

55. A kit comprising the pharmaceutical composition of any one of claims 45 to 54.

56. The kit of claim 55, further comprising instructions for administering the pharmaceutical composition.

57. A pharmaceutical composition as defined in any one of claims 45 to 54, for use in treating cancer.

58. The pharmaceutical composition for use of claim 57, wherein the cancer comprises a cancer cell which expresses B7-H4.

59. The pharmaceutical composition for use of claim 57 or 58, wherein the ADC is administered once every three weeks.

60. The pharmaceutical composition for use of any one of claims 57 to 59, wherein the cancer is selected from ovarian cancer, biliary tract cancer, breast cancer, pancreatic cancer, prostate cancer, hematological cancer, endometrial cancer, cholangiocarcinoma, NSCLC (squamous and/or adenocarcinoma), gastrointestinal cancer such as gastric cancer and colorectal cancer, and lung cancer.

61. The pharmaceutical composition for use of any one of claims 57 to 60, wherein the cancer is a breast cancer selected from hormone receptor-positive (HR+) breast cancer, human epidermal growth factor receptor 2 positive (HER2+) breast cancer, and triple negative breast cancer (TNBC).

62. The pharmaceutical composition for use of any one of claims 57 to 61, wherein the cancer is homologous recombination deficient (HRD) cancer.

63. The pharmaceutical composition for use of any one of claims 57 to 62, wherein the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L.

64. The pharmaceutical composition for use of claim 63, wherein the mutation in an HRD gene is selected from BRCA1, BRCA2, and ATM.

65. A method of treating cancer in a subject, comprising administering to the subject an antibody-drug conjugate (ADC) that specifically binds to B7-H4, the ADC comprising:
i. an antibody or antigen binding fragment thereof which binds to a B7-H4 polypeptide, comprising:
k) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
l) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
m) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
n) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
o) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof;
ii. a cleavable linker; and
iii. a cytotoxic agent;
wherein at least about 25% of cancer cells in the subject are B7-H4 positive cells.

66. A method for diagnosing a cancer sample as B7-H4 positive, comprising assaying the cancer sample, optionally using immunohistochemistry (IHC), to determine whether at least 25% of the cells express membranous B7-H4, further wherein optionally the IHC is performed with an antibody reagent.

67. The method of claim 65, wherein the B7-H4 positive cells are determined by measuring membranous B7-H4 expression prior to administration of the ADC, optionally wherein the B7-H4 expression is determined using immunohistochemistry (IHC), further wherein the IHC is optionally performed with an antibody reagent.

68. The method of claim 65, wherein the ADC is administered in an amount from about 0.8 mg/kg to about 4.8 mg/kg.

69. The method any one of claims 65 or 67 to 68, wherein the ADC is administered to the subject once every three weeks.

70. The method of claim 69, wherein the subject has been previously treated with a platinum-based chemotherapeutic agent.

71. The method of any one of claims 65-70, wherein the cancer is selected from ovarian cancer, biliary tract cancer, breast cancer, pancreatic cancer, prostate cancer, hematological cancer, endometrial cancer, cholangiocarcinoma, NSCLC (squamous and/or adenocarcinoma), gastrointestinal cancer such as gastric cancer and colorectal cancer, and lung cancer.

72. The method of any one of claims 65 to 71, wherein the cancer is a breast cancer selected from hormone receptor-positive (HR+) breast cancer, human epidermal growth factor receptor 2 positive (HER2+) breast cancer, and triple negative breast cancer (TNBC).

73. The method of any one of claims 65 to 72, wherein the cancer is homologous recombination deficient (HRD) cancer.

74. The method of claim 73, wherein the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L.

75. The method of claim 73, wherein the mutation in an HRD gene is selected from BRCA1, BRCA2, and ATM.

76. The method of any one of claims 65 or 67 to 75, wherein the antibody or antigen binding fragment thereof comprises a VH chain and a VL chain comprising the amino acid sequence of SEQ ID NO: 45 and SEQ ID NO: 34, respectively, or a functional variant thereof.

77. The method of any one of claims 65 or 67 to 76, wherein the antibody or antigen binding fragment thereof comprises a heavy chain comprising the amino acid sequence of SEQ ID NO: 51; and a light chain comprising the amino acid sequence of SEQ ID NO: 44.

78. The method of any one of claims 65 to 77, wherein the antibody or antigen binding fragment thereof is a monoclonal antibody.

79. The method of any one of claims 65 to 77, wherein the antibody or antigen binding fragment thereof is a humanized monoclonal antibody.

80. The method of any one of claims 65 to 79, wherein the cleavable linker is an mp-PEG8-val-ala linker.

81. The method of any one of claims 65 to 80, wherein the cytotoxic agent is a topoisomerase inhibitor.

82. The method of claim 81, wherein the topoisomerase inhibitor is a compound of Formula A*

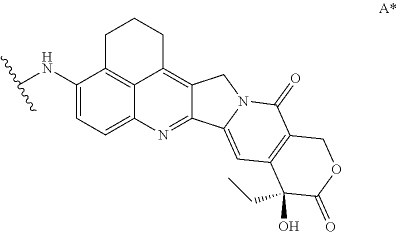 [SMILES: *Nc1ccc2nc3c(c4c2c1CCC4)Cn1c-3cc2c(c1=O)COC(=O)[C@]2(O)CC]

83. The method of any one of claims 65 to 81, wherein the ii) cleavable linker and iii) cytotoxic agent together are selected from the following compounds:

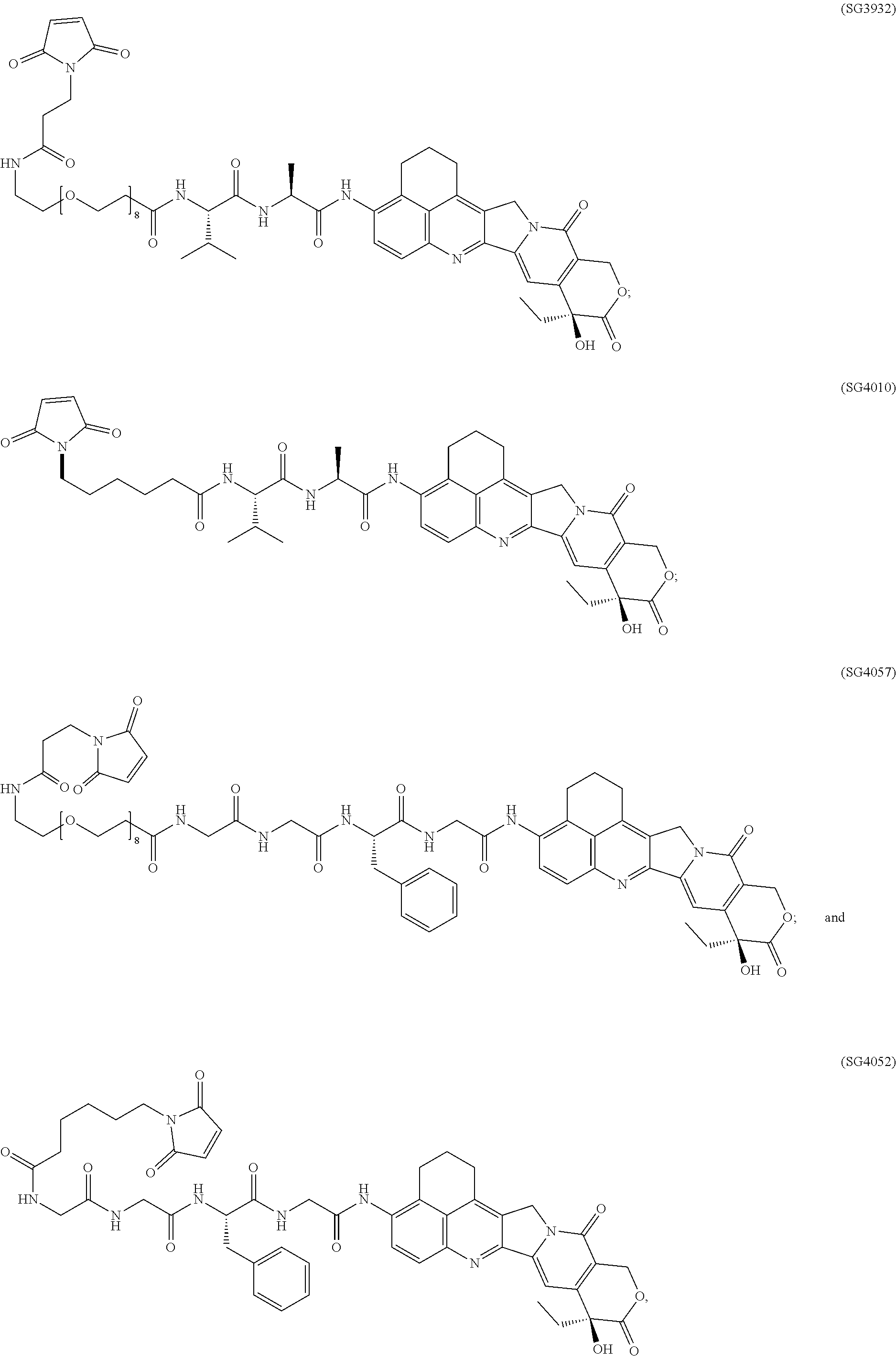

84. The method of claim 83, wherein the ii) linker and iii) cytotoxic agent together are the compound SG3932.

85. The method of any one of claims 65 to 84, wherein the ADC has a drug to antibody ratio (DAR) of between about 1 and about 8.

86. The method of any one of claims 65 to 84, wherein the ADC has a DAR of about 8.
